(19) United States
(12) Patent Application Publication
(10) Pub. No.: US 2020/0390681 A1
(43) Pub. Date: Dec. 17, 2020

(54) COSMETIC COMPOSITION TO IMPROVE THE RESISTANCE OF A HAIRSTYLE

(71) Applicant: Covestro Intellectual Property GmbH & Co. KG, Leverkusen (DE)

(72) Inventors: Sebastian Doerr, Düsseldorf (DE); Sophie Viala, Köln (DE); Laurence Pottie, Köln (DE)

(21) Appl. No.: 16/971,337

(22) PCT Filed: Feb. 25, 2019
(86) PCT No.: PCT/EP2019/054572

§ 371 Date: Aug. 20, 2020

(30) Foreign Application Priority Data
Feb. 27, 2018 (EP) 18158863.3

(51) Int. Cl. A61K 8/88; A61Q 5/06; A61K 8/87; A61K 8/34; A61K 8/86; A61K 8/36; A61K 8/40

(52) U.S. Cl. CPC A61K 8/88; A61Q 5/06; A61K 8/87; A61K 8/40; A61K 8/86; A61K 8/36; A61K 8/345

(57) ABSTRACT

The invention relates to a hairstyle-stabilizing composition comprising an especially preferred amorphous polyurethane urea having carboxyl or carboxylate groups. The invention further relates to the use of the especially preferred amorphous polyurethane urea for producing a hairstyle-stabilizing composition and also to a method for generating a water-stable hairstyle and a method for producing the hairstyle-stabilizing composition.

The invention relates to a hairstyle-stabilizing composition comprising an especially preferred amorphous polyurethane urea having carboxyl or carboxylate groups. The invention further relates to the use of the especially preferred amorphous polyurethane urea for producing a hairstyle-stabilizing composition and also to a method for generating a water-stable hairstyle and a method for producing the hairstyle-stabilizing composition.

Products known as hair fixatives are used for styling and stabilizing a variety of hairstyles. Hair fixatives are usually in the form of mousses or hairsprays of barely differing composition. Mousses are applied to damp hair as an aid for modeling the hairstyle. By way of contrast, hairsprays, hair creams, hair gels or hair waxes are applied in air to dry, already-styled hair to fix the hairstyle for everyday wear.

In the case of hairsprays and mousses, the means of fixing or styling the hairstyle usually takes the form of aerosol containers, squeeze bottles or preparations sprayable by pumping, spraying or foaming devices, which consist of an alcoholic, aqueous or aqueous-alcoholic solution of film-forming natural or synthetic polymers. These polymers may be selected from the group of non-ionic, cationic, amphoteric or anionic polymers.

Acrylate-based anionic or amphoteric polymers are commonly used as film-forming polymers in the prior art. The use of polyurethanes and polyurethane ureas as film formers is, however, also known. For example, WO 2009/118105 A1 describes hair-fixative compositions that obtain a polyurethane urea obtainable by reacting a water-insoluble, non-water-dispersible, isocyanate-functional polyurethane prepolymer with an amino-functional compound. The hair-fixative compositions disclosed therein are well suited for stabilizing hairstyles until the next hair wash. They do not, however, give hairstyles lasting shape that would survive a hair wash.

It was therefore an object of the present invention to at least partly overcome one disadvantage of the prior art.

Furthermore, it was an object to provide a specific composition that stabilizes the hairstyle, and is particularly suitable for achieving increased water resistance of the hairstyle thus shaped when used on hair. In particular, it was an object to achieve water resistance of the hairstyle shaped with the specific composition over several wash cycles, in particular to bring about water and wash resistance, which renders it unnecessary for the hair to be styled again after water contact or after washing over at least one, preferably several water contact or washing steps.

A further object was to provide a method which makes it possible to produce a water- and/or wash-resistant hairstyle, in particular to bring about a water and wash resistance, which renders it unnecessary for the hair to be styled again after water contact or after washing over at least one, preferably several water contact or washing steps.

Furthermore, it is an object of the invention to provide a method for producing the specific composition having the properties described above.

One object of the invention is to provide the use of the specific composition for generating waterproof hairstyles.

At least one of the objects is achieved by a composition according to the subject matter of claim 1 and also use thereof for generating waterproof hairstyles. Particular embodiments are described in the dependent claims. Another part of the objects is achieved by carrying out the method for producing the specific polyurethane urea.
[A1] A first subject matter of the invention relates to a hairstyle-stabilizing composition, comprising at least the following components:
   (V1) a preferably amorphous polyurethane urea (V1), which is obtainable by reacting at least
   A) one preferably aliphatic polyisocyanate component having an average isocyanate functionality of ≥1.8 and ≤2.6,
   B) one polymeric polyol component,
   C) one amino-functional chain extender component having at least 2 isocyanate-reactive amino groups, comprising at least one amino-functional compound C1) that does not have any ionic or ionogenic groups and/or an amino-functional compound C2) that has ionic or ionogenic groups,
   D) optionally further hydrophilizing components different from C2),
   E) optionally hydroxy-functional compounds having a molecular weight of 62 to 399 g/mol,
   F) optionally a compound having exactly one isocyanate-reactive group or a compound having more than one isocyanate-reactive group, where only one of the isocyanate-reactive groups reacts with the isocyanate groups present in the reaction mixture under the reaction conditions chosen, and
   G) optionally an aliphatic polyisocyanate component having an average isocyanate functionality of >2.6 and ≤4,
   wherein the polyurethane urea has at least one of the components C2) or D), and
   wherein the polyurethane urea has carboxyl groups or carboxylate groups;
   (V2) a polycarbodiimide component produced from aliphatic or cycloaliphatic polyisocyanates.

A hairstyle-stabilizing composition is understood to mean a composition which, after application to a user's hair and subsequent shaping of the hair into a hairstyle, makes it possible to achieve a higher water resistance or wash resistance than when shaping the hairstyle without applying the composition to the hair. In this case, the hairstyle-stabilizing composition can be applied to the hair as one component or as two components. If the hair is treated by applying the hairstyle-stabilizing composition in two steps, the polyurethane urea (V1) is first applied to the hair as a single component and in a subsequent step the polycarbodiimide component (V2) or vice versa. If the hair is treated by applying the hairstyle-stabilizing composition in one step, both components (V1) and (V2) are already mixed before the hair is treated.

According to the invention, water resistance is understood to mean that the resistance to water of the hairstyle shaped with the composition according to the invention is tested or determined. In these tests, the hair is at least partially covered with liquid water in a rinse treatment, that is to say soaked or rinsed therein. The water may be any form of liquid water that comes into contact with the hair in everyday life. Examples of this are drinking water, rainwater, swimming pool water (chlorinated, ozonized or kept microorganism-free by other means), seawater or distilled water.

Wash resistance in the context of the invention is understood to mean that the resistance to surfactant-containing water of the hairstyle shaped with the composition according to the invention is tested or determined, with the hair being completely covered by the surfactant-containing water during said washing treatment, that is to say rinsed therein.

Shaping of the hair into a hairstyle is preferably selected from the group consisting of:
a. straightening curly hair;
b. introducing curls into straight hair;
c. strengthening curls in already curled hair;
d. a combination of at least two of a. to c.

Straightening curly hair under a. may be done in any manner known to those skilled in the art, in particular by means of a straightening device. Curly hair is preferably straightened using a straightening iron, a straightening rod or a similar straightening device that is suitable for this purpose. The straightening device is preferably designed such that it is equipped with the aid of elements which may be heated to at least 150° C. The straightening device is preferably able to straighten curls to a degree of straightening of at least 50%, or preferably of at least 80%, or preferably of at least 90%. The degree of straightening is determined by measuring with a ruler the width of a tress of hair before and after straightening. A degree of straightening of 50%, 80% or 90% means that the width of the tress of hair after step a. has become respectively 50%, 80% or 90% narrower compared with the width before step a.

The introduction of curls into straight hair under b. may be done in any manner known to those skilled in the art, in particular by means of a curl-generating device. Straight hair is preferably made curly using a curling wand or a similar curl-generating device that is suitable for this purpose. The curl-generating device is preferably designed such that it includes the aid of curl-generating elements which may preferably be gently heated, for example to temperatures of 40 to 80° C. The curl-generating device is preferably suitable for creating curls with a degree of curl of at least 50%, or preferably at least 80%, or preferably at least 90%. The degree of curl is determined by measuring with a ruler the length of a tress of hair before and after introducing the curls. A degree of curl of 50%, 80% or 90% means that the length of the tress of hair after step b. is respectively 50%, 80% or 90% shorter than before step b.

The curls are preferably strengthened under c. in the same manner as introducing the curls under b. Alternatively or in addition, the curls can be strengthened under c. by means of the composition according to the invention in preferably damp hair, wherein the hair is subsequently dried with or without auxiliaries. The curl-generating device is preferably suitable for creating curls with a degree of curl of at least 30%, or preferably at least 40%, or preferably 50%. The degree of curl is determined by measuring with a ruler the length of a tress of hair before and after introducing the curls. A degree of curl of 30%, 40% or 50% means that the length of the tress of hair after step b. is respectively 30%, 40% or 50% shorter than before step c.

The shape of the styled hair is preferably retained to an extent of at least 5%, preferably at least 7%, or preferably at least 10% after contact with water. The degree of preservation of the shape of the styled hair can preferably vary substantially here, depending on the type of treatment. Thus, it makes a difference whether the hair had been straightened before use according to the invention of the composition according to the invention or whether the hair had been curled. The degree of preservation of the shape of the styled hair is referred to hereinbelow also as “styling retention”.

It is preferred to stabilize the hairstyle by means of the composition according to the invention by straightening the hair under a. to an extent of at least 50%, or preferably at least 60%, or preferably at least 70% or at least 80%, or at least 90% after contact with water, or the curl of the hair under b. to an extent of at least 5%, or preferably at least 7%, or preferably at least 10%, or the curl under c. to an extent of at least 5%, or preferably at least 7%, or preferably at least 10%.

The hairstyle-stabilizing composition preferably has a ratio by weight of the polyurethane urea (V1) to the polycarbodiimide component (V2) in a range from 10:1 to 1:2 or preferably in a range from 5:1 to 1:1.5, or preferably in a range from 4:1 to 1:1.

The reaction mixture of (V1) and (V2) in the production of the hairstyle-stabilizing composition preferably has a molar ratio of the carboxyl or carboxylate groups of (V1) to carbodiimide groups of (V2) in a range from 5:1 to 1:5, or preferably in a range from 2:1 to 1:2 or preferably in a range from 1.3:1 to 1:1.3.

The polyurethane urea (V1) of the composition is preferably amorphous. Amorphous in the context of this invention means that the polyurethane urea, within the temperature range specified in the test method detailed hereinafter, forms only such minor crystalline components, if any, that, by means of the DSC measurements described, it is possible to find only one or more glass transition points T_{g} but no fusion regions having an enthalpy of fusion ≥20 J/g within the temperature range mentioned.

The hairstyle-stabilizing composition preferably has a ratio by weight of the polyurethane urea (V1) to the polycarbodiimide component (V2) in a range from 80:1 to 1.2:1 or preferably in a range from 60:1 to 2:1, or preferably in a range from 40:1 to 4:1.

Preferably, amorphous polyurethane ureas in the context of the invention are polymeric compounds having at least two, preferably at least three, urethane group-containing repeating units:

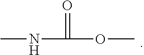

In accordance with the invention, the preferably amorphous polyurethane ureas, by virtue of their preparation, also have urea group-containing repeating units,

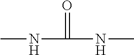

as formed particularly in the reaction of isocyanate-terminated prepolymers with amino-functional compounds.

Ionogenic groups in the context of this invention are understood to mean those functional groups that are capable of forming ionic groups, for example by neutralization with a base.

In accordance with the invention, the polyurethane urea (V1) has carboxyl groups or carboxylate groups. These can be introduced into the polyurethane urea by any of the components A) to G) used or other components used. The carboxyl groups or carboxylate groups are preferably introduced by component B).

The polyurethane urea (V1) of the composition preferably has a content of carboxyl groups and/or carboxylate groups in a range from 0.01 to 10% by weight, or preferably in a range from 0.02 to 8% by weight, or preferably in a range of 0.05 to 5% by weight, based on the total mass of the polyurethane urea.

Examples of components which comprise carboxyl groups or carboxylate groups and which can be used as constituent units for the preparation of (V1) are dimethylolpropionic acid, hydroxypivalic acid, natural and non-natural amino acids, such as 6-aminohexanoic acid, alanine, aspartic acid, glutamic acid, glutamine, glycine, lysine, leucine, isoleucine or mixtures of at least two thereof.

Component A) may be any polyisocyanate that the person skilled in the art would use for this purpose. Polyisocyanates suitable with preference as component A) are especially the preferred aliphatic polyisocyanates known per se to the person skilled in the art that have an average isocyanate functionality of ≥1.8 and ≤2.6. The term aliphatic also includes cycloaliphatic and/or araliphatic polyisocyanates.

Mean isocyanate functionality is understood here to mean the average number of isocyanate groups per molecule.

[A2] Preferred polyisocyanates are those in the molecular weight range from 140 to 336 g/mol. In a preferred embodiment of the hairstyle-stabilizing composition, the polyisocyanate of the polyisocyanate component A) is selected from the group consisting of toluene-2,4-diisocyanate (TDI), 2,2′-diphenylmethane diisocyanate (2,2′-MDI), 2,4′-diphenylmethane diisocyanate (2,4′-MDI), 4,4′-diphenylmethane diisocyanate (4,4′-MDI), 1,4-diisocyanatobutane (BDI), 1,5-pentane diisocyanate, (PDI), 1,6-diisocyanatohexane (HDI), 1,3-bis (isocyanatomethyl)benzene (1,3-xylylene diisocyanate, XDI), 1,4-bis(isocyanatomethyl)benzene (1,4-xylylene diisocyanate, XDI), 1,3-bis(1-isocyanato-1-methylethyl)benzene (TMXDI), 1,4-bis(1-isocyanato-1-methylethyl)benzene (TMXDI), 4-isocyanatomethyl-1,8-octane diisocyanate (trisisocyanatononane (TIN)), 2-methyl-1,5-diisocyanatopentane, 1,5-diisocyanato-2,2-dimethylpentane, 2,2,4- or 2,4,4 trimethyl-1,6-diisocyanatohexane, 1,10-diisocyanatodecane and the cycloaliphatic diisocyanates 1,3- and 1,4-diisocyanatocyclohexane, 1,4-diisocyanato-3,3,5-trimethylcyclohexane, 1,3 -diisocyanato-2(4)-methylcyclohexane, 1-isocyanato-3,3,5 -trimethyl-5 -isocyanatomethylcyclohexane (isophorone diisocyanate, IPDI), 1-isocyanato-1-methyl-4(3)-isocyanatomethylcyclohexane, 1,8-diisocyanato-p-menthane, 4,4′-diisocyanato-1,1′-bi(cyclohexyl), 4,4′-diisocyanato-3,3′-dimethyl-1,1′-bi(cyclohexyl), 4,4′-diisocyanato-2,2′,5,5′-tetramethyl-1,1′-bi (cyclohexyl), 4,4′- and/or 2,4″-diisocyanatodicyclohexylmethane, 4,4′- diisocyanato-3,3′-dimethyldicyclohexylmethane, 4,4′-diisocyanato-3,3′,5,5′-tetramethyldicyclohexylmethane, 1,3-diisocyanatoadamantane, and 1,3-dimethyl-5,7-diisocyanatoadamantane or any mixtures of such isocyanates. The polyisocyanates are especially preferably selected from 1,4-butylene diisocyanate, 1,5-pentamethylene diisocyanate (PDI), 1,6-hexamethylene diisocyanate (HDI), isophorone diisocyanate (IPDI), 2,2,4- and/or 2,4,4-trimethylhexamethylene-diisocyanate, the isomeric bis(4,4′-isocyanatocyclohexyl)methanes or mixtures thereof of any isomer content (H12-MDI), 1,4-cyclohexylene diisocyanate, 4-isocyanatomethyl-1,8-octane diisocyanate (nonane triisocyanate) and alkyl 2,6-diisocyanatohexanoates (lysine diisocyanates) with C1-C8-alkyl groups or mixtures of at least two thereof.

The present invention preferably relates to the composition according to the invention, wherein the polyisocyanate A) is selected from the group consisting of 1,4-butylene diisocyanate, 1,6-hexamethylene diisocyanate (HDI), 2,2,4 and/or 2,4,4-trimethylhexamethylene diisocyanate, the isomeric bis(4,4′-isocyanatocyclohexy)methanes or mixtures of any isomer content thereof, 1,4-cyclohexylene diisocyanate, 1,4-phenylene diisocyanate, 2,4- and/or 2,6-toluylene diisocyanate, 1,5-naphthylene diisocyanate, 2,2′- and/or 2,4′- and/or 4,4′-diphenylmethane diisocyanate, 1,3- and/or 1,4-bis(2-isocyanatoprop-2-yl)benzene (TMXDI), 1,3-bis(isocyanatomethyl)benzene (XDI), alkyl 2,6-diisocyanatohexanoate (lysine diisocyanates) having C1-C8-alkyl groups, and 4-isocyanatomethyl-1,8-octane diisocyanate (nonane triisocyanate) and triphenylmethane-4,4′,4″-triisocyanate or a mixture of at least two thereof.

As well as the aforementioned polyisocyanates, it is also possible to use modified diisocyanates having an average isocyanate functionality of ≥2 and ≤2.6, with uretdione, isocyanurate, urethane, allophanate, biuret, iminooxadiazinedione or oxadiazinetrione structure, and mixtures of proportions of these and/or the above.

Preference is given to polyisocyanates or polyisocyanate mixtures of the aforementioned type having exclusively aliphatically or cycloaliphatically bonded isocyanate groups or mixtures of these and an average NCO functionality of the mixture of ≥1.8 and ≤2.6 and more preferably ≥2.0 and ≤2.4.

[A3] In a preferred embodiment of the hairstyle-stabilizing composition, component A) comprises an aliphatic or cycloaliphatic polyisocyanate. The polyisocyanate of the polyisocyanate component A) is preferably selected from the group consisting of HDI, IPDI and/or H12-MDI or modification products thereof, especially preferably selected from HDI and/or IPDI.

In an especially preferred variant, IPDI and HDI are present in a mixture as component A).

The weight ratio of IPDI:HDI for the polyisocyanate component A) is preferably within a range from 1.05 to 10, more preferably within a range from 1.1 to 5, and most preferably within a range from 1.1 to 1.5.

In a preferred embodiment, the preferably amorphous polyurethane urea used in accordance with the invention is prepared using >5% and ≤40% by weight of component A) and more preferably ≥10% and ≤35% by weight of component A), based in each case on the total mass of the preferably amorphous polyurethane urea.

Further preferably, the preferably amorphous polyurethane urea is also prepared using component G), an aliphatic polyisocyanate component having an average isocyanate functionality (average number of isocyanate groups per molecule) of >2.6 and ≤4, preferably ≥2.8 and ≤3.8. Component G) is preferably used in this case in a mixture with component A).

Particularly suitable components G) are oligomeric diisocyanates having a functionality of >2.6 and ≤4, preferably ≥2.8 and ≤3.8, having isocyanurate, urethane, allophanate, biuret, iminooxadiazinedione or oxadiazinetrione structure. G) especially preferably comprises isocyanurate structures.

The aliphatic polyisocyanate component G) preferably consists of an aliphatic or cycloaliphatic polyisocyanate oligomer based on HDI, IPDI and/or H12-MDI, especially preferably based on HDI.

The molar ratio of NCO groups of component A) to component G) is preferably from 100:0.5 to 100:50; particularly preferably from 100:2 to 100:15 and especially preferably from 100:3 to 100:8.

The preferably amorphous polyurethane urea used in accordance with the invention is preferably prepared using ≥0% and ≤10% by weight of component G) and more preferably ≥0.1% and ≤3% by weight of component G), based in each case on the total mass of the preferably amorphous polyurethane urea.

Suitable di- or higher-functional polymeric polyols B) are compounds having at least two isocyanate-reactive hydrogen atoms and an average molecular weight, preferably in a range from 400 to 10 000 g/mol, or preferably from 500 to 8000 g/mol, or preferably from 1000 to 5000 g/mol, determined by gel permeation chromatography against polystyrene standard in tetrahydrofuran at 23° C. Examples of suitable structural components are polyethers, polyesters, polycarbonates, polylactones and polyamides. Preferred polyols B) have 2 to 4, particularly preferably 2 to 3 hydroxyl groups. Mixtures of various such compounds are also possible.

Suitable polyester polyols are in particular linear polyester diols or also sparsely branched polyester polyols, as can be produced in a known manner from aliphatic, cycloaliphatic or aromatic di- or polycarboxylic acids such as succinic acid, methylsuccinic acid, glutaric acid, adipic acid, pimelic acid, suberic acid, azelaic acid, sebacic acid, nonanedicarboxylic acid, decanedicarboxylic acid, terephthalic acid, isophthalic acid, o-phthalic acid, tetrahydrophthalic acid, hexahydrophthalic acid, cyclohexanedicarboxylic acid, maleic acid, fumaric acid, malonic acid or trimellitic acid and acid anhydrides such as o-phthalic, trimellitic or succinic anhydride or mixtures thereof with polyhydric alcohols, such as ethanediol, di-, tri-, tetraethylene glycol, 1,2-propanediol, di-, tri-, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, 1,8-octanediol, 1,10-decanediol, 1,12-dodecanediol or mixtures thereof, optionally with the use of higher-functional polyols such as trimethylolpropane, glycerol or pentaerythritol. Cycloaliphatic and/or aromatic di- and polyhydroxyl compounds are of course also suitable as polyhydric alcohols for producing the polyester polyols. Instead of the free polycarboxylic acid, the corresponding polycarboxylic anhydrides or corresponding polycarboxylic esters of lower alcohols or mixtures thereof can also be used to produce the polyesters.

Self-evidently, the polyester polyols can also be homopolymers or copolymers of lactones, which are obtained preferably by adding lactones or lactone mixtures, such as butyrolactone, ε-caprolactone and/or methyl-ε-caprolactone, to the suitable di- and/or higher-functional starter molecules, such as the low molecular weight, polyhydric alcohols mentioned above as structural components for polyester polyols. Preference is given to the corresponding polymers of ε-caprolactone.

Particular preference is given to the largely linear polyesterpolyols, which comprise adipic acid and 1,4-butanediol and/or 1,6-hexanediol and/or 2,2-dimethyl-1,3-propanediol as structural components.

Polycarbonates comprising hydroxyl groups are also suitable as polyhydroxyl components, for example those which can be produced by reacting diols, such as 1,4-butanediol and/or 1,6-hexanediol, with diaryl carbonates such as diphenyl carbonate, dialkyl carbonates such as dimethyl carbonate, or phosgene. The at least partial use of polycarbonates comprising hydroxyl groups can improve the hydrolysis resistance of the polyurethane or polyurethane urea dispersions.

Preference is given to polycarbonates which are prepared by reacting 1,6-hexanediol with dimethyl carbonate.

Suitable polyether polyols are, for example, the polyaddition products of styrene oxides, of ethylene oxide, propylene oxide, tetrahydrofuran, butylene oxide, epichlorohydrin, and the mixed addition and grafting products thereof, and the polyether polyols obtained by condensation of polyhydric alcohols or mixtures of the same and those obtained by alkoxylation of polyhydric alcohols, amines and amino alcohols. Polyether polyols suitable as polyol components B) are homopolymers, copolymers and graft polymers of propylene oxide and of ethylene oxide, which are accessible by addition of the epoxides mentioned to low molecular weight diols or triols, as are mentioned above as structural components for polyester polyols, or to higher-functional low molecular weight polyols such as pentaerythritol or sugar or to water.

Particularly preferred difunctional or higher-functional polyols B) are polyester polyols, polylactones and polycarbonates.

The polymeric polyether polyols preferably used as component B) preferably have number-average molecular weights in a range from 400 to 8000 g/mol, preferably in a range from 600 to 6000 g/mol, or preferably in a range from 1000 to 3000 g/mol, determined by gel permeation chromatography against polystyrene standard in tetrahydrofuran at 23° C., and/or an OH functionality of preferably in a range from 1.5 to 6, or preferably in a range from 1.8 to 3, or preferably in a range from 1.9 to 2.1. The expression “polymeric” polyetherpolyols here means more particularly that the polyols mentioned have at least two, preferably at least three, repeating units bonded to one another.

The number-average molecular weight for the purposes of this specification is always determined by gel permeation chromatography (GPC) in tetrahydrofuran at 23° C. The procedure is in accordance with DIN 55672-1: “Gel permeation chromatography, Part 1—Tetrahydrofuran as eluent” (SECurity GPC System from PSS Polymer Service, flow rate 1.0 ml/min; columns: 2× PSS SDV linear M, 8×300 mm, 5 μm; RID detector). Polystyrene samples of known molar mass are used for calibration. The number-average molecular weight is calculated with software support. Baseline points and evaluation limits are fixed according to DIN 55672 Part 1.

Suitable polyetherpolyols are, for example, the addition products, known per se, of styrene oxide, ethylene oxide, propylene oxide, butylene oxide and/or epichlorohydrin onto di- or polyfunctional starter molecules. Polyalkylene glycols in particular, such as polyethylene glycols, polypropylene glycols and/or polybutylene glycols, are thus employable, especially with the abovementioned preferred molecular weights. Suitable starter molecules that may be used are all compounds known from the prior art, for example water, butyldiglycol, glycerol, diethylene glycol, trimethylolpropane, propylene glycol, sorbitol, ethylenediamine, triethanolamine, butane-1,4-diol.

[A4] In a preferred embodiment of the hairstyle-stabilizing composition, component B) comprises poly(propylene glycol) polyether polyols. Preferably, the hairstyle-stabilizing composition includes poly(propylene glycol) polyetherpolyols in a range from 50% to 100% by weight, or preferably in a range from 70% to 100% by weight or preferably in a range from 90% to 100% by weight, particularly preferably to an extent of 100% by weight, based in each case on the total weight of component B).

If component B) has more than 50% by weight poly(propylene glycol) polyether polyols, based on the sum of all constituents of component B), then optionally a small amount of component F) is used or preferably component F) is not used at all. If component B) for producing the hairstyle-stabilizing composition has more than 10% by weight, preferably more than 20% by weight, or preferably more than 30% by weight, based on the sum of all constituents of component B), of poly(tetramethylene) polyether polyols, it is preferred that component F) is present.

[A5] In a preferred embodiment of the hairstyle-stabilizing composition, component B) has an average molecular mass in a range from 400 to 4000 g/mol, or preferably in a range from 500 to 3500 g/mol, or preferably in a range from 800 to 3000 g/mol.

[A6] In a preferred embodiment of the composition, component B) comprises or consists of a mixture of poly(propylene glycol) polyether polyols having different average molecular weight, where the poly(propylene glycol) polyether polyols differ in their number-average molecular weights by at least 100 g/mol, preferably by at least 200 g/mol, or preferably by at least 400 g/mol, or preferably by at least 800 g/mol, or preferably by at least 1000 g/mol. Preferably, the number-average molecular weights of the poly(propylene glycol) polyether polyols differ by not more than 5000 g/mol, or by not more than 4000 g/mol, or by not more than 3000 g/mol.

Component B) preferably comprises a mixture of poly(propylene glycol) polyether polyols I having a number-average molecular weight Mn of ≥400 and ≤1500 g/mol, particularly preferably of ≥600 and ≤1200 g/mol, especially preferably of 1000 g/mol, and poly(propylene glycol) polyether polyols II having a number-average molecular weight Mn of ≥1500 and ≤8000 g/mol, particularly preferably of ≥1800 and ≤3000 g/mol, especially preferably of 2000 g/mol.

The weight ratio of the poly(propylene glycol) polyether polyols I to the poly(propylene glycol) polyether polyols II is preferably in the range from 0.01 to 10, particularly preferably in the range from 0.02 to 5, especially preferably in the range from 0.05 to 1.

[A7] In a further preferred embodiment of the hairstyle-stabilizing composition, the polyol B) is selected from the group consisting of succinic acid, methylsuccinic acid, glutaric acid, adipic acid, pimelic acid, suberic acid, azelaic acid, sebacic acid, nonanedicarboxylic acid, decanedicarboxylic acid, terephthalic acid, isophthalic acid, o-phthalic acid, tetrahydrophthalic acid, hexahydrophthalic acid, cyclohexanedicarboxylic acid, maleic acid, fumaric acid, malonic acid or trimellitic acid and acid anhydrides such as o-phthalic, trimellitic or succinic anhydride or mixtures thereof with polyhydric alcohols, such as ethanediol, di-, tri-, tetraethylene glycol, 1,2-propanediol, di-, tri-, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, 1,8-octanediol, 1,10-decanediol, 1,12-dodecanediol or a mixture of at least two thereof.

In the production of the preferably amorphous polyurethane urea, an amino-functional chain extender component C) having at least 2 isocyanate-reactive amino groups is used, comprising at least one amino-functional compound C1) that does not have any ionic or ionogenic groups and/or an amino-functional compound C2) that has ionic or ionogenic groups.

The amino-functional compounds of component C) component are preferably selected from primary and/or secondary diamines. More particularly, the amino-functional compounds C) comprise at least one diamine.

[A8] In a preferred embodiment of the hairstyle-stabilizing composition, the amino-functional component C) comprises at least one amino-functional compound C2) that has ionic and/or ionogenic groups. The amino-functional compound C2) is mandatory if component D) is not present in the aqueous polyurethane urea dispersion for the preparation of the hairstyle-stabilizing composition according to the invention.

The amino-functional component C) preferably comprises both amino-functional compounds C2) having an ionic and/or ionogenic group and amino-functional compounds C1) having no ionic or ionogenic group.

For example, components C1) used may be organic di- or polyamines, for example ethylene-1,2-diamine, 1,2- and 1,3-diaminopropane, 1,4-diaminobutane, 1,6-diaminohexane, isophoronediamine (IPDA), isomeric mixture of 2,2,4- and 2,4,4-trimethylhexamethylenediamine, 2-methylpentamethylenediamine, diethylenetriamine, 4,4-diaminodicyclohexylmethane and/or dimethylethylenediamine or mixtures of at least two of these.

Preferably, component C1) is selected from the group consisting of ethylene-1,2-diamine, bis(4-aminocyclohexyl)methane, 1,4-diaminobutane, IPDA, ethanolamine, diethanolamine and diethylenetriamine or a mixture of at least two of these.

Preferably, component C1) comprises >75 mol %, particularly preferably ≥80 mol %, especially preferably ≥85 mol %, further preferably ≥95 mol % and still further preferably 100 mol % ethylene-1,2-diamine or IPDA or a mixture of ethylene-1,2-diamine and IPDA, where the sum total of the two amines in relation to the total amount of C1) is preferably in the proportions mentioned for component C1).

Preferably, the hydrophilizing component C2) comprises at least one anionically hydrophilizing compound. Further preferably, the hydrophilizing component C2) includes an anionically hydrophilizing compound to an extent of at least 80% by weight, or preferably to an extent of at least 90% by weight, based on the total weight of component C2). More preferably, component C2) consists of exclusively anionically hydrophilizing compounds.

Suitable anionically hydrophilizing compounds comprise at least one anionic or ionogenic group that can be converted to an anionic group. Further preferably, suitable anionically hydrophilizing compounds have at least two amino groups and more preferably two amino groups. More preferably, the hydrophilizing component C2) comprises or consists of an anionically hydrophilizing compound having at least one anionic or ionogenic group and at least two amino groups.

Suitable anionically hydrophilizing compounds as component C2), also called hydrophilizing agents C2) hereinafter, preferably comprise a sulfonic acid or sulfonate group, more preferably a sodium sulfonate group. Suitable anionically hydrophilizing compounds as component C2) are especially the alkali metal salts of mono- and diaminosulfonic acids. Examples of such anionic hydrophilizing agents are salts of 2-(2-aminoethylamino)ethanesulfonic acid, N-(propyl or butyl)ethylenediaminesulfonic acid or propylene-1,2- or -1,3-diamine-β-ethylsulfonic acid or mixtures of at least two of these.

Particularly preferred anionic hydrophilizing agents C2) are those that comprise sulfonate groups as ionic groups and two amino groups, such as the salts of 2-(2-aminoethylamino)ethylsulfonic acid and propylene-1,3-diamine-β-ethylsulfonic acid. Very particular preference is given to using 2-(2-aminoethylamino)ethylsulfonic acid or salts thereof as anionic hydrophilizing agent C2).

The anionic group in component C2) may optionally also be a carboxylate or carboxylic acid group. Examples of carboxylic acids are 6-aminohexanoic acid, alanine, aspartic acid, glutamic acid, glutamine, glycine, lysine, leucine and isoleucine or a mixture of at least two of these. Further examples of suitable substances for component C2) are preferably selected from diaminocarboxylic acids. Lysine is an example of diaminocarboxylic acids.

In this alternative embodiment, however, the carboxylic acid-based components C2) have to be used in higher concentrations compared to those components C2) bearing sulfonate or sulfonic acid groups. More preferably, therefore, the preferably amorphous polyurethane urea is prepared using no hydrophilizing compounds bearing exclusively carboxylate groups as anionic groups of component C2).

The preferably amorphous polyurethane urea used in accordance with the invention is preferably prepared using ≥0.1% and ≤10% by weight of component C2) and more preferably ≥0.5% and ≤4% by weight of component C2), based in each case on the total mass of the preferably amorphous polyurethane urea.

Hydrophilization can also be accomplished using mixtures of anionic hydrophilizing agents C2) and further hydrophilizing agents D) that are different than C2).

Suitable further hydrophilizing agents D) are, for example, non-ionic hydrophilizing compounds D1) and/or hydroxy-functional ionic or ionogenic hydrophilizing agents D2).

[A9] In a preferred embodiment of the hairstyle-stabilizing composition, component D) is exclusively non-ionically hydrophilizing component D1).

Suitable hydroxy-functional ionic or ionogenic hydrophilizing agents as component D2) are, for example, hydroxycarboxylic acids such as mono- and dihydroxycarboxylic acids, such as 2-hydroxyacetic acid, 3-hydroxypropanoic acid, 12-hydroxy-9-octadecanoic acid (ricinoleic acid), hydroxypivalic acid, lactic acid, dimethylolbutyric acid and/or dimethylolpropionic acid or mixtures of at least two of these. Preference is given to hydroxypivalic acid, lactic acid and/or dimethylolpropionic acid, particular preference to dimethylolpropionic acid. Preference is given to using no hydroxy-functional ionic or ionogenic hydrophilizing agents D2), especially preferably no hydrophilizing agents having carboxylate and hydroxyl groups, for example dimethylolpropionic acid. Preferably, the amount of hydroxy-functional ionic or ionogenic hydrophilizing agents D2) present in the preferably amorphous polyurethane urea is in a range from 0% to 1% by weight, or preferably in a range from 0.01% to 0.5% by weight, based on the total mass of the preferably amorphous polyurethane urea.

Suitable non-ionically hydrophilizing compounds as component D1) are, for example, polyoxyalkylene ethers having isocyanate-reactive groups, such as hydroxyl, amino or thiol groups.

Preference is given to monohydroxy-functional polyalkylene oxide polyether alcohols having a statistical average of 5 to 70, preferably 7 to 55, ethylene oxide units per molecule, such as are accessible in a manner known per se by alkoxylation of suitable starter molecules (for example in Ullmann's Encyclopedia of Industrial Chemistry, 4th Edition, Volume 19, Verlag Chemie, Weinheim pp 31-38). These are either pure polyethylene oxide ethers or mixed polyalkylene oxide ethers and they comprise at least 30 mol %, preferably at least 40 mol % ethylene oxide units, based on all alkylene oxide units present.

Particularly preferred nonionic compounds are monofunctional mixed polyalkylene oxide polyethers having 40 to 100 mol % of ethylene oxide units and 0 to 60 mol % of propylene oxide units.

Suitable starter molecules for such non-ionic hydrophilizing agents are especially saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or olein alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and heterocyclic secondary amines such as morpholine, pyrrolidine, piperidine or 1H-pyrazole. Preferred starter molecules are saturated monoalcohols of the abovementioned type. It is particularly preferable to use diethylene glycol monobutyl ether, methanol or n-butanol as starter molecules.

Alkylene oxides suitable for the alkoxylation reaction are especially ethylene oxide and propylene oxide, which can be used in the alkoxylation reaction in any sequence or else in a mixture.

The preferably amorphous polyurethane urea comprises ≥0 and ≤20% by weight of component D), preferably ≥0.1 and ≤10% by weight of component D) and especially preferably ≥1 and ≤5% by weight of component D), based in each case on the total mass of the preferably amorphous polyurethane urea. In a further preferred embodiment, component D) is not used for preparing the preferably amorphous polyurethane urea. In this case, the presence of component C2) is mandatory in the polyurethane urea.

As component E) it is optionally possible to use polyols, especially non-polymeric polyols, of said molecular weight range from 62 to 399 g/mol having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), trimethylolpropane, trimethylolethane, glycerol, pentaerythritol and any desired mixtures thereof with one another.

The preferably amorphous polyurethane urea preferably has ≤10% by weight of component E), or preferably ≤5% by weight of component E), based in each case on the total mass of the preferably amorphous polyurethane urea. The preferably amorphous polyurethane urea preferably includes component E) in a range from 0.1 to 10% by weight, or preferably in a range from 0.2 to 8% by weight, or preferably in a range from 0.1 to 5% by weight, based in each case on the total mass of the preferably amorphous polyurethane urea. In a further preferred embodiment, component E) is not used for preparing the preferably amorphous polyurethane urea.

To produce the preferably amorphous polyurethane urea, preference is given to using ≥0.5 and ≤20% by weight formed from the sum of the components C1) and optionally E) and particularly preferably ≥1 and ≤15% by weight formed from the sum of the components C1) and optionally E), based in each case on the total mass of the preferably amorphous polyurethane urea.

Di- or higher-functional polyol components having a molecular weight of 62 to 399 which are suitable as component E) are, for example, the products listed under B), provided they have a molecular weight of 62 to 399 g/mol. Other suitable components are the polyhydric, especially dihydric alcohols mentioned for producing the polyester polyols, and furthermore low molecular weight polyester diols such as adipic acid bis(hydroxyethyl) esters or short-chain homoaddition and mixed addition products of ethylene oxide or propylene oxide started on aromatic diols. Examples of aromatic diols, which may be used as starters for short-chain homopolymers and copolymers of ethylene oxide or propylene oxide, are for example 1,4-, 1,3-, 1,2-dihydroxybenzene or 2,2-bis(4-hydroxyphenyl)propane (bisphenol A).

Monofunctional isocyanate-reactive compounds containing hydroxyl groups can also be used as component F). Examples of such monofunctional compounds are ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol, which may also be used as mixtures of at least two of the above. The hairstyle-stabilizing composition preferably comprises less than 10% by weight of component F), or preferably less than 5% by weight of component F), or preferably in a range from 0.01 to 5% by weight, in each case based on the total mass of the hairstyle-stabilizing composition, more preferably component F) is not used to produce the hairstyle stabilizing composition.

The hairstyle-stabilizing composition preferably comprises one or more cosmetic auxiliary substances V3) that are customary in cosmetics, such as antioxidants, light stabilizers and/or other auxiliaries and additives, for example emulsifiers, wetting agents such as interface-active substances, plasticizers such as glycerol, glycol and phthalate esters and ethers, defoamers, thickeners and rheology modifiers, gelling agents, antisticking agents, surfactants, active ingredients, humectants, fillers, UV filters, film formers, solvents, coalescents, aromas, odor absorbers, fragrances and perfumes, gel formers and/or other polymer dispersions, for example dispersions based on polyacrylates, pigments, dyes, anticorrosives, neutralizing agents, leveling agents and/or thixotropic agents, suppleness promoters, preservatives, proteins and derivatives thereof, amino acids, vitamins, opacifiers, stabilizers, sequestering agents, complexing agents, pearlescent agents, esthetic enhancers, fatty acids, fatty alcohols, triglycerides, botanical extracts and clarifying agents. The compositions according to the invention preferably have one or more emulsifiers or surface-active agents.

The amounts of the various additives are known to the person skilled in the art for the range of the hair cosmetic to be used, particularly of the hairstyle-stabilizing compositions, and are for example in the range from 0% to 25% by weight, preferably from 0% to 15% by weight, or preferably from 0.001% to 15% by weight, or preferably for each individual additive within a range from 0.001% to 5% by weight, or in each case preferably from 0.01% to 3% by weight, based on the total weight of the composition.

The hairstyle-stabilizing composition preferably further includes at least one of the following components:
V3) at least one cosmetically active substance and/or one or more cosmetic auxiliary substances,
V4) at least one solvent or thinner,
V5) at least one additional film former.

For instance, preferred hairstyle-stabilizing compositions in the form of oil-in-water emulsions (O/W) preferably comprise at least one emulsifier with an HLB value >7 and optionally a coemulsifier.

The following non-ionic emulsifiers are used advantageously:
a) partial fatty acid esters and fatty acid esters of polyhydric alcohols and ethoxylated derivatives thereof (e.g. glyceryl monostearate, sorbitan stearate, glyceryl stearyl citrate, sucrose stearate)
b) ethoxylated fatty alcohols and fatty acids.

Particularly advantageous non-ionic O/W emulsifiers are ethoxylated fatty alcohols or fatty acids, preferably PEG-100 stearate, PEG-40 stearate, ceteareth-20, ceteth-20, steareth-20, ceteareth-12, ceteth-12, steareth-12 and esters of mono-, oligo- or polysaccharides with fatty acids, preferably cetearyl glucoside, methyl glucose distearate.

Advantageous anionic emulsifiers are soaps (for example sodium or triethanolamine salts of stearic acid or palmitic acid) and esters of citric acid such as glyceryl stearate citrate.

Suitable coemulsifiers used for O/W emulsions of the invention may be fatty alcohols having 8 to 30 carbon atoms, monoglyceryl esters of saturated or unsaturated, branched or unbranched alkanecarboxylic acids having a chain length of 8 to 24 carbon atoms, especially 12 to 18 carbon atoms, propylene glycol esters of saturated or unsaturated, branched or unbranched alkanecarboxylic acids having a chain length of 8 to 24 carbon atoms, especially 12 to 18 carbon atoms, and sorbitan esters of saturated or unsaturated, branched or unbranched alkanecarboxylic acids having a chain length of 8 to 24 carbon atoms, especially 12 to 18 carbon atoms.

Particularly advantageous coemulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol, and polyethylene glycol (2) stearyl ether (steareth-2).

It may be advantageous in the context of the present invention to use further emulsifiers. This may be done, for example, to further increase the water resistance of the formulations of the invention. Examples of suitable emulsifiers are alkyl methicone copolyols and alkyl dimethicone copolyols, in particular cetyl dimethicone copolyol, lauryl methicone copolyol, W/O emulsifiers such as sorbitan stearate, glyceryl stearate, glycerol stearate, sorbitan oleate, lecithin, glyceryl isostearate, polyglyceryl-3 oleate, polyglyceryl-3 diisostearate, PEG-7 hydrogenated castor oil, polyglyceryl-4 isostearate, acrylate/C₁₀-30 alkyl acrylate crosspolymer, sorbitan isostearate, poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoylpolyglyceryl-3 diisostearate, glycol distearate, and polyglyceryl-3 dipolyhydroxy stearate.

The hairstyle-stabilizing compositions preferably comprise thickeners, especially in the water phase, for example in an O/W composition. Advantageous thickeners are:
crosslinked or non-crosslinked acrylic or methacrylic acid homo- or copolymers. These include crosslinked homopolymers of methacrylic acid or acrylic acid, copolymers of acrylic acid and/or methacrylic acid and monomers derived from other acrylic or vinyl monomers, such as C10-30 alkyl acrylates, C10-30 alkyl methacrylates and vinyl acetate.
thickening polymers of natural origin, for example based on cellulose, guar gum, xanthan, scleroglucan, gellan gum, rhamsan and karaya gum, alginates, maltodextrin, starch and its derivatives, locust bean gum, hyaluronic acid, carrageenan.
non-ionic, anionic, cationic or amphoteric associative polymers, for example based on polyethylene glycols and their derivatives, or polyurethanes.
crosslinked or noncrosslinked homopolymers or copolymers based on acrylamide or methacrylamide, such as homopolymers of 2-acrylamido-2-methylpropanesulfonic acid, copolymers of acrylamide or methacrylamide and methacryloyloxyethyltrimethylammonium chloride or copolymers of acrylamide and 2-acrylamido-2-methylpropanesulfonic acid.

Particularly advantageous thickeners are thickening polymers of natural origin, crosslinked acrylic acid or methacrylic acid homo- or copolymers and crosslinked copolymers of 2-acrylamido-2-methylpropanesulfonic acid.

Very particularly advantageous thickeners are xanthan gum, such as the products supplied under the names Keltrol® and Kelza® by CP Kelco or the products from RHODIA with the name Rhodopol® and guar gum, such as those obtainable under the name Jaguar® HP105 by RHODIA.

Very particularly advantageous thickeners are also crosslinked homopolymers of methacrylic acid or acrylic acid, which are obtainable commercially from Lubrizol under the names Carbopol® 940, Carbopol® 941, Carbopol® 980, Carbopol® 981, Carbopol® ETD 2001, Carbopol® EDT 2050, Carbopol® 2984, Carbopol® 5984 and Carbopol® Ultrez 10, from 3V under the names Synthalen® K, Synthalen® L and Synthalen® MS, and from PROTEX under the names Modarez® V 1250 PX, Modarez® V2000 PX, Viscaron® A1600 PE and Viscaron® A700 PE.

Very particular advantageous thickeners are crosslinked copolymer of acrylic acid or methacrylic acid and a C₁₀₋₃₀-alkyl acrylate or C₁₀₋₃₀-alkyl methacrylate and copolymers of acrylic acid or methacrylic acid and vinylpyrrolidone. Such copolymers are obtainable commercially, for example, from Lubrizol under the names Carbopol® 1342, Carbopol® 1382, Pemulen® TR1 or Pemulen® TR2 and from ISP under the names Ultrathix® P-100 (INCI: Acrylic Acid/VP Crosspolymer).

Very particularly advantageous thickeners are crosslinked copolymers of 2-acrylamido-2-methylpropanesulfonic acid. Such copolymers are obtainable, for example, from Clariant under the names Aristoflex® AVC (INCI: Ammonium Acryloyldimethyltaurate/VP Copolymer).

If the thickeners are used, the thickeners are generally present at a concentration of about 0% to 2% by weight, preferably 0% to 1% by weight or preferably within a range from 0.01% to 1% by weight, based on the total weight of the composition according to the invention.

The preferred solvents as component V4) are, for example, the aliphatic alcohols having C1-4 carbon atoms, such as ethanol and isopropanol; polyol and derivatives thereof such as propylene glycol, dipropylene glycol, butylene 1,3-glycol, polypropylene glycol, glycol ethers such as alkyl (C1-4) ethers of mono-, di- or tripropylene glycol or mono-, di- or triethylene glycol, and mixtures thereof.

The proportion of the solvent or solvents V4) in the composition according to the invention may, for example, be in the range from 0% to 25% by weight, or preferably 0% to 15% by weight, based on the total weight of the composition.

The compositions of the invention may further contain a propellant gas.

Preferred propellants are hydrocarbons such as propane, isobutane and n-butane and mixtures thereof. Compressed air, carbon dioxide, nitrogen, nitrogen dioxide and dimethyl ether and mixtures of all these gases can likewise be used.

Of course, the person skilled in the art is aware that there are propellants that are non-toxic per se and would in principle be suitable for the implementation of the present invention in the form of aerosol preparations, but which should nevertheless be dispensed with because of a harmful effect on the environment or other accompanying circumstances. These are especially fluorocarbons and chlorofluorocarbons (CFCs) such as 1,2-difluoroethane (propellant 152 A).

The hairstyle-stabilizing composition preferably also comprises hair-care active ingredients. Care substances used with preference may be cyclic polydimethylsiloxanes (cyclomethicones) or silicone surfactants (polyether-modified siloxanes) of the dimethicone copolyol or simethicone type. Cyclomethicones are supplied, inter alia, under the trade names Abil® K4 by Goldschmidt or, for example, DC 244, DC 245 and DC 345 by Dow Corning. Dimethicone copolyols are supplied, for example, under the DC 193 trade name by Dow Corning or Belsil® DM 6031 by Wacker.

### Surfactants

The compositions of the invention may also contain surfactants selected from the group of anionic, cationic, non-ionic and/or amphoteric surfactants.

Advantageous anionic surfactants in the context of the present invention are:
acylamino acids and salts thereof, such as acylglutamates, especially sodium acylglutamate, and sarcosinates, for example myristoylsarcosine, TEA-lauroyl sarcosinate, sodium lauroyl sarcosinate and sodium cocoyl sarcosinate,
sulfonic acids and salts thereof, such as acyl isethionate, for example sodium or ammonium cocoylisethionate, sulfosuccinates, for example sodium dioctyl sulfosuccinate, disodium laureth sulfosuccinate, disodium lauryl sulfosuccinate and disodium undecyleneamido MEA sulfosuccinate, disodium PEG-5 lauryl citrate sulfosuccinate and derivatives;
sulfuric esters, such as alkyl ether sulfate, for example sodium, ammonium, magnesium, MIPA and TIPA laureth sulfate, sodium myrethsulfate and sodium C₁₂ to C₁₃-parethsulfate, and alkyl sulfates, for example sodium, ammonium and TEA lauryl sulfate,
taurates, for example, sodium lauroyl taurate and sodium methyl cocoyl taurate;
ether carboxylic acids, for example sodium laureth-13 carboxylate and sodium PEG-6 cocamide carboxylate, sodium PEG-7 olive oil carboxylate;
phosphoric esters and salts, such as DEA-oleth-10 phosphate and dilaureth-4 phosphate;
alkylsulfonates, for example sodium cocomonoglyceride sulfate, sodium C₁₂ to C₁₄ olefinsulfonate, sodium lauryl sulfoacetate and magnesium PEG-3 cocamide sulfate,
acylglutamates, such as di-TEA-palmitoyl aspartate and sodium caprylic/capric glutamate;
acyl peptides, for example, palmitoyl hydrolyzed milk protein, sodium cocoyl hydrolyzed soy protein, and sodium/potassium cocoyl hydrolyzed collagen;
carboxylic acids and derivatives, for example lauric acid, aluminum stearate, magnesium alkanoate and zinc undecylenate, ester carboxylic acids, for example, calcium stearoyl lactylate, laureth-6 citrate, and sodium PEG-4 lauramide carboxylate;
alkylaryl sulfonates.

Advantageous cationic surfactants in the context of the present invention are quaternary surfactants. Quaternary surfactants contain at least one nitrogen atom covalently bonded to 4 alkyl or aryl groups. Advantageous examples include alkyl betaine, alkylamidopropyl betaine and alkylamidopropyl hydroxysultaine.

Further advantageous cationic surfactants for the purposes of the present invention are also alkylamines, alkylimidazoles and ethoxylated amines and especially salts thereof.

Advantageous amphoteric surfactants for the purposes of the present invention are acyl/dialkylethylenediamines, for example sodium acylamphoacetate, disodium acylamphodipropionate, disodium alkylamphodiacetate, sodium acylamphohydroxypropylsulfonate, disodium acylamphodiacetate, sodium acylamphopropionate, and N-coconut fatty acid amidoethyl-N-hydroxyethylglycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Advantageous active non-ionic surfactants for the purposes of the present invention are alkanolamides, such as cocamide MEA/DEA/MIPA, esters formed by esterification of carboxylic acids with ethylene oxide, glycerol, sorbitan or other alcohols, ethers, for example ethoxylated alcohols, ethoxylated lanolin, ethoxylated polysiloxanes, propoxylated POE ethers, alkyl polyglycosides, such as lauryl glucoside, decyl glycoside and cocoglycoside, glycosides with an HLB value of at least 20 (e.g. Belsil® SPG 128V from Wacker).

Further advantageous non-ionic surfactants are alcohols and amine oxides, such as cocoamidopropylamine oxide.

Among the alkyl ether sulfates, sodium alkyl ether sulfates based on di- or triethoxylated lauryl and myristyl alcohol are especially preferred. They clearly surpass the alkyl sulfates with regard to insensitivity to water hardness, thickenability, cold solubility and, in particular, skin and mucous membrane compatibility. Lauryl ether sulfate has better foaming properties than myristyl ether sulfate, but is inferior in terms of mildness.

Alkyl ether carboxylates with moderate and especially with higher are among the mildest surfactants that exist, but have poor foaming and viscosity characteristics. They are often used in combination with alkyl ether sulfates and amphoteric surfactants.

Sulfosuccinic esters (sulfosuccinates) are mild and readily foaming surfactants but are preferably used only together with other anionic and amphoteric surfactants on account of their poor thickenability, and preferably only in neutral or well-buffered products on account of their low hydrolysis stability.

Amidopropyl betaines have excellent skin and eye mucous membrane compatibility. In combination with anionic surfactants, their mildness can be synergistically improved. Preference is given to the use of cocamidopropyl betaine.

Amphoacetates/amphodiacetates, being amphoteric surfactants, have very good skin and mucous membrane compatibility and can have a conditioning effect or increase the care effect of additives. They are used in a similar manner to the betaines for optimization of alkyl ether sulfate formulations. Most preferred are sodium cocoamphoacetate and disodium cocoamphodiacetate.

Alkyl polyglycosides are mild and have good universal properties, but poor foaming For this reason, they are preferably used in combination with anionic surfactants.

### Conditioners

Optionally, the compositions of the invention contain a conditioner. Preferred conditioners are, for example, all compounds which are listed in the International Cosmetic Ingredient Dictionary and Handbook (Volume 4, publisher: R. C. Pepe, J. A. Wenninger, G. N. McEwen, The Cosmetic, Toiletry, and Fragrance Association, 9th edition, 2002) under Section 4 under the keywords Hair Conditioning Agents, Humectants, Skin Conditioning Agents, Skin Conditioning Agents-Emollient, Skin Conditioning Agents-Humectant, Skin Conditioning Agents-Miscellaneous, Skin Conditioning

Agents-Occlusive and Skin Protectants and all the compounds listed in EP-A 934 956 (pp. 11-13) under “water soluble conditioning agent” and “oil soluble conditioning agent”.

Particular advantageous conditioners are, for example, the compounds referred to as Polyquaternium according to INCI (especially Polyquaternium-1 to Polyquaternium-114).

Suitable conditioners include, for example, polymeric quaternary ammonium compounds, cationic cellulose derivatives, chitosan derivatives, guar gum derivatives and polysaccharides, especially guar hydroxypropylammonium chloride (e.g. Jaguar® Excel, Jaguar® 162 from Rhodia).

Further conditioners that are advantageous in accordance with the invention are non-ionic poly-N-vinylpyrrolidone/polyvinyl acetate copolymers (e.g. Luviskol® VA 64 from BASF AG), anionic acrylate copolymers (e.g. Luviflex® Soft from BASF AG), and/or amphoteric amide/acrylate/methacrylate copolymers (e.g. Amphomer® from National Starch). Further possible conditioners are quaternized silicones.

Optionally, conventional additives may likewise be included in the composition, for example to impart certain modifying properties thereto. These may be, for instance, silicones or silicone derivatives, wetting agents, humectants, plasticizers such as glycerol, glycol and phthalic esters and ethers, odorants and perfumes, UV absorbers, dyes, pigments and other colorants, anticorrosive agents, neutralizing agents, antioxidants, antisticking agents, combining agents and conditioners, antistats, shine agents, preservatives, proteins and derivatives thereof, amino acids, vitamins, emulsifiers, surface-active agents, viscosity modifiers, thickeners and rheology modifiers, gelling agents, opacifiers, stabilizers, surfactants, sequestrants, complexing agents, pearlescent agents, esthetic enhancers, fatty acids, fatty alcohols, triglycerides, botanical extracts, clarifying aids and film formers.

These additives are generally present at a concentration of about 0.001% to 15% by weight, preferably 0.01% to 10% by weight, based on the total weight of the composition.

At least one additional film former of component V5) selected from the group consisting of a non-ionic, an anionic, an amphoteric and/or a cationic polymer is preferably present in the hairstyle-stabilizing composition.

Preferably, the film former V5) is selected from the group consisting of a polyacrylate, a polyacrylamide, a polyurethane, a polyurea, a polysiloxane or a mixture of at least two of these. The polyacrylates also include silicone-acrylate copolymers.

To improve the resistance of decorative products to water, tears or sweat (often called water resistance) among other reasons, film-forming polymers are used as film formers.

Preferred film-forming polymers selected are polymers based on acrylates or vinylpyrrolidones. Advantageous film formers are trimethylsiloxysilicates, silicone acrylate copolymers (e.g. TIB4-200 from Dow Corning or KP-561 from Shin Etsu), trimethyl pentaphenyl trisiloxanes (Dow Corning 555 Cosmetic Fluid from Dow Corning Ltd.) or vinylpyrrolidone copolymer (e.g. PVP/eicosene copolymer or PVP/hexadecane copolymer).

Preference is generally given to using non-ionic, anionic, amphoteric and/or cationic polymers as film formers. Preferably non-ionic polymers that are used in the hairstyle-stabilizing composition, either alone or in combination, preferably with anionic and/or amphoteric and/or zwitterionic polymers, are preferably selected from the group consisting of:
polyalkyloxazolines;
vinyl acetate homo- or copolymers, including, for example, copolymers of vinyl acetate and acrylic esters, copolymers of vinyl acetate and ethylene, copolymers of vinyl acetate and maleic esters;
acrylic ester copolymers, for example the copolymers of alkyl acrylate and alkyl methacrylate, copolymers of alkyl acrylate and urethanes;
copolymers of acrylonitrile and non-ionic monomer selected from butadiene and (meth)acrylate;
styrene homo- and copolymers, including, for example, homopolystyrene, copolymers of styrene and alkyl (meth)acrylate, copolymers of styrene, alkyl methacrylate and alkyl acrylate, copolymers of styrene and butadiene, copolymers of styrene, butadiene and vinylpyridine;
polyamides;
vinyllactam homo- or copolymers, such as vinylpyrrolidone homo- or copolymers, including, for example, polyvinylpyrrolidone, polyvinylcaprolactam, copolymers of N-vinylpyrrolidone and vinyl acetate and/or vinyl propionate in various concentration ratios, polyvinylcaprolactam, polyvinylamides and salts thereof, and also copolymers of vinylpyrrolidone and dimethylaminoethyl methacrylate, terpolymers of vinylcaprolactam, vinylpyrrolidone and dimethylaminoethyl methacrylate;
polysiloxanes;
homopolymers of N-vinylformamide such as PVF from AkzoNobel;
polyimides such as polyimide-1 in Styleze™ XT3 from Ashland, and
polyurethanes such as Baycusan® C2000 from Covestro Deutschland AG; or
mixtures of at least two of the aforementioned.

Particularly preferred non-ionic polymers are acrylic ester copolymers, homopolymers of vinylpyrrolidone and copolymers, and polyvinylcaprolactam.

Particularly preferred non-ionic polymers are homopolymers of vinylpyrrolidone, e.g. Luviskol K from BASF, copolymers of vinylpyrrolidone and vinyl acetate, e.g. Luviskol® VA products from BASF or PVPVA® S630L from ISP, terpolymers of vinylpyrrolidone, vinyl acetate and propionate, e.g. Luviskol® VAP from BASF, and polyvinylcaprolactams, e.g. Luviskol® PLUS from BASF.

Advantageous anionic polymers are homo- or copolymers with monomer units which contain acid groups and have optionally been copolymerized with comonomers not containing acid groups. Suitable monomers are unsaturated, free-radically polymerizable compounds having at least one acid group, and especially carboxylic acid, sulfonic acid or phosphonic acid.

Examples of anionic polymers containing carboxylic acid groups are:
acrylic acid or methacrylic acid homo- or copolymers or salts thereof. These include, for example, the copolymers of acrylic acid and acrylamide and/or sodium salts thereof, copolymers of acrylic acid and/or methacrylic acid and an unsaturated monomer selected from the group of ethylene, styrene, vinyl esters, acrylic esters, methacrylic esters, optionally ethoxylated compounds, copolymers of vinylpyrrolidone, acrylic acid and C1-C20 alkyl methacrylates, e.g. Acrylidone® LM from ISP, copolymers of methacrylic acid, ethyl acrylate and tert-butyl acrylate, e.g. Luvimer® 100 P from BASF;
crotonic acid derivative homo- or copolymers or salts thereof. These include, for example, vinyl acetate/crotonic acid, vinyl acetate/acrylate and/or vinyl acetate/vinyl neodecanoate/crotonic acid copolymers, for example Resyn® 28-1310 or Resyn® 28-2930 from AkzoNobel or Luviset® CAN from BASF, sodium acrylate/vinyl alcohol copolymers;
unsaturated C4-C8 carboxylic acid derivatives or carboxylic anhydride copolymers selected from copolymers of maleic acid/maleic anhydride or fumaric acid/fumaric anhydride or itaconic acid/itaconic anhydride and at least one monomer selected from the group of vinyl esters, vinyl ethers, vinyl halogen derivatives, phenyl vinyl derivatives, acrylic acid, acrylic esters or copolymers of maleic acid/maleic anhydride or fumaric acid/fumaric anhydride or itaconic acid/itaconic anhydride and at least one monomer selected from the group of allyl esters, methallyl esters and optionally acrylamides, methacrylamides, alpha-olefin, acrylic esters, methacrylic esters, vinylpyrrolidones. Further preferred polymers are methyl vinyl ethers/maleic acid copolymers obtained through hydrolysis of vinyl ether/maleic anhydride copolymers. These polymers may also be partly esterified (ethyl, isopropyl or butyl esters) or partly amidated;
water-soluble or -dispersible anionic polyurethanes, e.g. Luviset.® PUR from BASF, and DynamX® from AkzoNobel, Baycusan® C1000, Baycusan® C1001, Baycusan® C1003, Baycusan® C1004, Baycusan® C1008 from Covestro Deutschland AG.

This preceding list should be considered nonexhaustive and hence nonlimiting.

Advantageous anionic polymers containing sulfonic acid groups are salts of polyvinylsulfonic acids, polystyrenesulfonic acids, for example sodium polystyrenesulfonate, or polyacrylamidosulfonic acids.

Particularly advantageous anionic polymers are acrylic acid copolymers, crotonic acid derivative copolymer, copolymers of maleic acid/maleic anhydride or fumaric acid/fumaric anhydride or itaconic acid/itaconic anhydride and at least one monomer selected from the group of vinyl esters, vinyl ethers, vinyl halogen derivatives, phenyl vinyl derivatives, acrylic acid, acrylic esters and salts of polystyrenesulfonic acids.

Very particularly advantageous anionic polymers are acrylate copolymers, e.g. Luvimer® from BASF, ethyl acrylate/N-tert-butylacrylamide/acrylic acid copolymers ULTRAHOLD® STRONG from BASF, VA/crotonate/vinyl neodecanoate copolymer, e.g. Resyn® 28-2930 from AkzoNobel, copolymers such as copolymers of methyl vinyl ether and partially esterified maleic anhydride, e.g. GANTREZ® from Ashland and sodium polystyrene sulfonates such as Flexan® 130 from AkzoNobel.

Advantageous amphoteric polymers may be selected from the polymers containing units A and B distributed randomly in the polymer chain, where A is a unit derived from a monomer having at least one basic nitrogen atom, and B represents a unit that originates from an acidic monomer having one or more carboxylic or sulfonic acid groups. Alternatively, A and B may be groups derived from zwitterionic carboxybetaine monomers or sulfobetaine monomers. A and B may also be a cationic polymer chain containing primary, secondary, tertiary or quaternary groups, where at least one amino group bears a carboxyl group or sulfonic acid group bonded via a hydrocarbyl group, or A and B are part of a polymer chain with an ethylene-α,β-dicarboxylic unit in which the carboxylic acid groups have been reacted with a polyamine containing one or more primary or secondary amino groups.

Preferred amphoteric polymers are:
polymers which are formed in the copolymerization of a monomer derived from a vinyl compound having a carboxyl group, such as, more particularly, acrylic acid, methacrylic acid, maleic acid, a-chloroacrylic acid, and a basic monomer. The basic monomer is derived from a vinyl compound which is substituted and contains at least one basic atom, such as, more particularly, dialkylaminoalkyl methacrylate and acrylate, dialkylaminoalkylmethacrylamide and -acrylamide. Compounds of this kind have been described in American patent U.S. Pat. No. 3,836,537.
Polymers having units derived from: a) at least one monomer selected from the acrylamides or methacrylamides, substituted by an alkyl group on the nitrogen atom, b) at least one acidic comonomer containing one or more reactive carboxyl groups, and c) at least one basic comonomer, such as esters of acrylic acid and methacrylic acid with primary, secondary, tertiary and quaternary amino substituents and the quaternization product of dimethylaminoethyl methacrylate with dimethyl sulfate or diethyl sulfate.
Particularly preferred N-substituted acrylamides or methacrylamides are compounds wherein the alkyl groups comprise 2 to 12 carbon atoms. Very particular preference is given to N-ethylacrylamide, N-t-butylacrylamide, N-t-octylacrylamide, N-octylacrylamide, N-decylacrylamide, N-dodecylacrylamide and the corresponding methacrylamides.
Suitable acidic comonomers are especially selected from the group of acrylic acid, methacrylic acid, crotonic acid, itaconic acid, maleic acid, fumaric acid and the alkyl monoesters having 1 to 4 carbon atoms of maleic acid, maleic anhydride, fumaric acid or fumaric anhydride.
Preferred basic comonomers are aminoethyl methacrylate, butylaminoethyl methacrylate, N,N-dimethylaminoethyl methacrylate, N-t-butylaminoethyl methacrylate.
Crosslinked and wholly or partly acylated polyaminoamides derived from polyaminoamides of the following general formula:

—[CO—R—CO—Z]—
where R is a divalent group derived from a saturated dicarboxylic acid, an aliphatic mono- or dicarboxylic acid having an ethylenic double bond, an ester of these acids with a lower alkanol having 1 to 6 carbon atoms, or a group which forms in the addition of one of these acids onto a bis-primary or bis-secondary amine, and Z is a group derived from a bis-primary, mono- or bis-secondary polyalkylenepolyamine, and preferably: a) as a quantitative fraction of 60 to 100 mol % the —NH—[(CH₂)ₓ—NH—]p- groups where x=2 and p=2 or 3 or x=3 and p=2, the group being formed by diethylenetriamine, triethylenetetramine or dipropylenetriamine; b) as a quantitative fraction of 0 to 40 mol % the —NH—[(CH₂)ₓ—NH—]p- group where x=2 and p=1, which is obtainable from ethylenediamine, or the group that originates from piperazine:

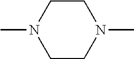

c) in a quantitative fraction of 0 to 20 mol % the —H—(CH₂)₆—NH— group obtainable from hexamethylenediamine, these polyaminoamides being crosslinked by addition of a bifunctional crosslinker, preferably selected from the group of epihalohydrins, diepoxides, dianhydrides and bis-unsaturated derivatives, in an amount within a range from 0.025 to 0.35 mol of crosslinker per amino group of the polyaminoamide, and acylated with acrylic acid, chloroacetic acid or an alkanesulfone or salts thereof.
The saturated carboxylic acids are preferably from the acids having 6 to 10 carbon atoms, such as adipic acid, 2,2,4-trimethyladipic acid and 2,4,4-trimethyladipic acid, terephthalic acid; acids having an ethylenic double bond, for example acrylic acid.
The alkane sultones used in the acylation are preferably propane sultone or butane sultone; the salts of acylating agents are preferably the sodium salts or potassium salts.
Polymers having zwitterionic units of the following formula:

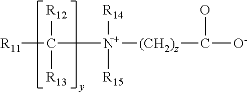

in which R₁₁ is a polymerizable unsaturated group, such as acrylate, methacrylate, acrylamide or methacrylamide, y and z are integers from 1 to 3, R₁₂ and R₁₃ are a hydrogen atom, methyl, ethyl or propyl, R₁₄ and R₁₅ are a hydrogen atom or an alkyl group chosen such that the sum total of the carbon atoms R₁₄ and R₁₅ does not exceed 10.
Polymers containing such units may also have units that originate from non-zwitterionic monomers, such as dimethyl- and diethylaminoethyl acrylate or dimethyl and diethylaminoethyl methacrylate or alkyl acrylates or alkyl methacrylates, acrylamides or methacrylamides or vinyl acetate.
Polymers which are derived from chitosan and contain monomer units conforming to the following formulae:

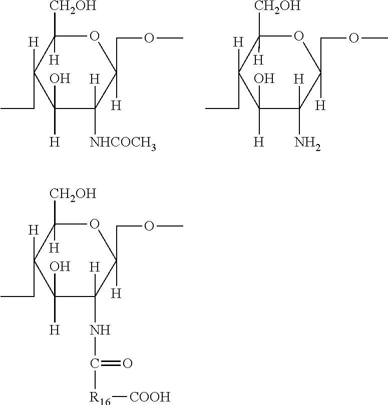

where the first unit is present in proportions of 0% to 30%, the second unit in proportions of 5% to 50% and the third unit in proportions of 30% to 90%, with the proviso that R₁₆ in the third unit is a group of the following formula:

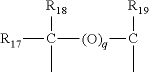

in which, if q=0, the R₁₇, R₁₈ and R₁₉ groups are the same or different and are each a hydrogen atom, methyl, hydroxyl, acetoxy or amino, a monoalkylamine radical or a dialkylamine radical which is optionally interrupted by one or more nitrogen atoms and/or optionally by one or more amino groups, hydroxyl groups, carboxyl groups, alkylthio groups, sulfo groups, alkylthio groups wherein the alkyl group bears an amino radical, where at least one of the R₁₇, R₁₈ and R₁₉ groups in this case is a hydrogen atom; or, if q=1, the R₁₇, R₁₈ and R₁₉ groups are each a hydrogen atom, and the salts that form these compounds with bases or acids.
Polymers which conform to the following general formula and are described, for example, in French Patent 1 400 366:

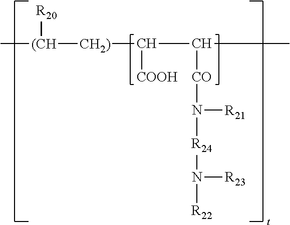

in which R₂₀ is a hydrogen atom, CH₃O, CH₃CH₂O or phenyl, R₂₁ is a hydrogen atom or a lower alkyl group, such as methyl or ethyl, R₂₂ is a hydrogen atom or a lower C₁₋₆-alkyl group, such as methyl or ethyl, R₂₃ is a lower C₁₋₆-alkyl group, such as methyl or ethyl, or a group of the formula: —R₂₄—N(R₂₂)₂ where R₂₄ is a —CH₂—CH₂, —CH₂—CH₂—CH₂— or —CH₂—CH(CH₃)— group and where R₂₂ has the definitions given above.
Polymers which can be formed in the N-carboxyalkylation of chitosan, such as N-carboxymethylchitosan or N-carboxybutylchitosan.
   Alkyl(C₁₋₅) vinyl ether/maleic anhydride copolymers which have been partly modified partly by semiamidation with an N,N-dialkylaminoalkylamine such as N,N-dimethylaminopropylamine or an N,N-dialkylamino alcohol. These polymers may also contain further comonomers, such as vinylcaprolactam.

Examples of very particularly advantageous amphoteric polymers are the copolymers octylacrylamide/acrylates/butylaminoethyl methacrylate copolymer, which are sold under the names AMPHOMER®, AMPHOMER® LV 71 or BALANCE® 47 by AkzoNobel, and methyl methacrylate/methyl dimethylcarboxymethylammonioethylmethacrylate copolymers.

It may be advantageous to neutralize the anionic and amphoteric polymers with suitable bases to improve their water solubility or their water dispersibility.

Neutralizing agents used for polymers containing acid groups may be the following bases: Hydroxides wherein the cation is ammonium or an alkali metal, for example NaOH or KOH.

Other neutralizing agents are primary, secondary or tertiary amines, amino alcohols or ammonia. Preference is given here to using 2-amino-2-methylpropane-1,3 -diol (AMPD), 2-amino-2-ethylpropane-1,3-diol (AEPD), 2-amino-2-methyl-1-propanol (AMP), 2-amino-1-butanol (AB), 2-aminopropane-1,3-diol, monoethanolamine (MEA), diethanolamine (DEA), triethanolamine (TEA), monoisopropanolamine (MIPA), diisopropanolamine (DIPA), triisopropanolamine (TIPA), Dimethyl Laurylamine (DML), Dimethyl Myristalamine (DMM), and Dimethyl Stearamine (DMS).

The neutralization may be partial or complete according to the end use.

It is optionally possible to use, albeit less preferably, cationic polymers, for example polymers containing primary, secondary, tertiary and/or quaternary amino groups that are part of the polymer chain or bonded directly to the polymer chain.

The additional film former V5) is preferably a polyurethane which is obtainable by reacting one or more water-insoluble, non-water-dispersible, isocyanate-functional polyurethane prepolymers with one or more amino-functional compounds B), where B) can be selected from the same compounds as described for B) above.

In the context of the invention, the term “water-insoluble, non-water-dispersible polyurethane prepolymer” means more particularly that the water solubility of the prepolymer used in accordance with the invention at 23° C. is less than 10 g/liter, preferably less than 5 g/liter, and the prepolymer at 23° does not result in a sedimentation-stable dispersion in water, especially deionized water. In other words, the prepolymer settles out when an attempt is made to disperse it in water.

Preferably, the NCO-terminated polyurethane prepolymer is obtainable from the reaction of a reaction mixture comprising a polyisocyanate and polyol. The polyisocyanate preferably has a functionality in a range from >1.5 to 6, or preferably from 1.8 to 5, or preferably from 2 to 4, especially of 2. Suitable polyisocyanates are aliphatic, aromatic araliphatic or cycloaliphatic polyisocyanates. Examples of such suitable polyisocyanates are butylene 1,4-diisocyanate, hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof of any isomer content, cyclohexylene 1,4-diisocyanate, 4-isocyanatomethyloctane 1,8-diisocyanate (nonane triisocyanate), phenylene 1,4-diisocyanate, toluylene 2,4- and/or 2,6-diisocyanate, naphthylene 1,5-diisocyanate, diphenylmethane 2,2′- and/or 2,4′- and/or 4,4′-diisocyanate, 1,3- and/or 1,4-bis(2-isocyanatoprop-2-yl)benzene (TMXDI), 1,3-bis(isocyanatomethyl)benzene (XDI) and alkyl 2,6-diisocyanatohexanoates (lysine diisocyanates) with C1 to C8 alkyl groups.

Preferably, the polyisocyanate is an aliphatic polyisocyanate. Preferred aliphatic diisocyanates are hexamethylene diisocyanate and isophorone diisocyanate and mixtures thereof.

The polyol preferably has a functionality of >1.5 to 6 or preferably from 1.8 to 5, or preferably from 2 to 4, especially of 2. Preferably, the polyol is selected from the group consisting of a polyether polyol, a polycarbonate polyol, a polyether polycarbonate polyol, a polyester polyol or a mixture of at least two of these. Preferably, the polyol includes a polyol containing polyoxyethylene groups. With regard to the polyols, preference is given to copolymers of ethylene oxide and propylene oxide having an ethylene oxide content, based on the total amount of the oxyalkylene groups present, of 60 to 85 mol %. Preference is also given to polyester polyols preferably formed from an acid selected from the group consisting of malonic acid, succinic acid, glutaric acid, adipic acid, pimelic acid, suberic acid, azelaic acid, sebacic acid or a mixture of at least two of these with a polyol selected from the group consisting of ethane-1,2-diol, propane-1,3-diol, 2,2-dimethylpropane-1,3-diol (neopentanediol), butane-1,4-diol, 2,2-dimethylbutane-1,4-diol, pentane-1,5-diol, 2,2-dimethylbutane-1,4-diol, hexane-1,6-diol or a mixture of at least two of these.

Preferably, the total amount of carbodiimides and the total amount of film formers present in the hairstyle-stabilizing composition is in a ratio from 2:1 to 1:4, or preferably in a ratio from 1.5:1 to 1:3, or preferably in a ratio from 1:1 to 1:3. The hairstyle-stabilizing composition preferably includes a higher percentage by weight of film formers than of compounds comprising at least three carbodiimide groups, also called polycarbodiimide.

The hairstyle-stabilizing composition is preferably in a form selected from the group consisting of a spray, in particular a pump spray, an aerosol, a gel, a foam, a mousse, a lotion, a wax, in particular a hair wax, a pomade, an oil, a milk, an oil in water emulsion, an aqueous solution or a cream. The hairstyle-stabilizing composition is preferably in the form of a pump spray, an aerosol, a gel, a foam, a mousse, a lotion, a wax, a pomade.

The composition of the invention may further comprise a wax.

In the context of the present document, a wax is defined as a lipophilic fatty substance that is solid at room temperature (25° C.) and shows a reversible solid/liquid change of state at a melting temperature between 30° C. and 200° C. Above the melting point, the viscosity of the wax is low and it becomes miscible with oils.

The wax is advantageously chosen from the groups of natural waxes, for example cotton wax, carnauba wax, candelilla wax, esparto wax, japan wax, montan wax, sugarcane wax, beeswax, wool wax, shellac, microwaxes, ceresin, ozokerite, ouricury wax, cork fiber wax, lignite waxes, berry wax, shea butter, or synthetic waxes such as paraffin waxes, polyethylene waxes, waxes produced by Fischer-Tropsch synthesis, hydrogenated oils, fatty acid esters and glycerides that are solid at 25° C., silicone waxes and derivatives (alkyl derivatives, alkoxy derivatives and/or esters of polymethylsiloxane) and mixtures thereof. The waxes can be in the form of stable dispersions of colloidal wax particles which can be produced by known processes, for example according to “Microemulsions Theory and Practice”, L. M. Prince Ed., Academic Press (1977), pages 21-32.

The waxes may be present in amounts of 0% to 10% by weight, based on the total weight of the composition, and preferably 0% to 5% by weight.

The cosmetically acceptable medium of the composition according to the invention preferably comprises water and optionally a cosmetically acceptable water-miscible suitable organic solvent.

The water used in the composition according to the invention may be a blossom water, pure demineralized water, mineral water, thermal water, and/or seawater.

In the case of an O/W composition as the composition according to the invention, the water content may be in the range from 40% to 95% by weight, preferably in the range from 50% to 90% by weight, most preferably in the range from 60% to 80% by weight, based on the total weight of the composition. In the case of a W/O composition, the water content is in the range from 0% to 60% by weight, preferably in the range from 10% to 50% by weight, most preferably in the range from 30% to 50% by weight, based on the total weight of the composition.

The composition may also be foamed with a propellant gas. The emulsions described above may be stabilized by O/W, W/O or W/Si emulsifiers, thickeners (such as hydrodispersion) or solids (for example a Pickering emulsion).

The composition may contain one or more emulsifiers or surface-active agents.

Thus, oil-in-water emulsions (O/W) in particular preferably contain at least one emulsifier having an HLB value >7 and optionally a coemulsifier.

The following non-ionic emulsifiers are used advantageously:
a) fatty acid partial esters and fatty acid esters of polyhydric alcohols and ethoxylated derivatives thereof (for example glyceryl monostearate, sorbitan stearate, glyceryl stearyl citrate, sucrose stearate)
b) ethoxylated fatty alcohols and fatty acids.

Particularly advantageous non-ionic O/W emulsifiers are ethoxylated fatty alcohols or fatty acids, preferably PEG-100 stearate, PEG-40 stearate, ceteareth-20, ceteth-20, steareth-20, ceteareth-12, ceteth-12, steareth-12 and esters of mono-, oligo- or polysaccharides with fatty acids, preferably cetearyl glucoside, methyl glucose distearate.

Advantageous anionic emulsifiers are soaps (for example sodium or triethanolamine salts of stearic acid or palmitic acid) and esters of citric acid such as glyceryl stearate citrate.

Suitable coemulsifiers used for O/W emulsions according to the invention may be fatty alcohols having 8 to 30 carbon atoms, monoglyceryl esters of saturated or unsaturated, branched or unbranched alkanecarboxylic acids having a chain length of 8 to 24 carbon atoms, especially 12 to 18 carbon atoms, propylene glycol esters of saturated or unsaturated, branched or unbranched alkanecarboxylic acids having a chain length of 8 to 24 carbon atoms, especially 12 to 18 carbon atoms, and sorbitan esters of saturated or unsaturated, branched or unbranched alkanecarboxylic acids having a chain length of 8 to 24 carbon atoms, especially 12 to 18 carbon atoms.

Particularly advantageous coemulsifiers are glyceryl monostearate, glyceryl monooleate, diglyceryl monostearate, sorbitan monoisostearate, sucrose distearate, cetyl alcohol, stearyl alcohol, behenyl alcohol, isobehenyl alcohol, and polyethylene glycol (2) stearyl ether (steareth-2).

It may be advantageous in the context of the present invention to use further emulsifiers. This may be done, for example, to further increase the water resistance of the formulations of the invention. Examples of suitable emulsifiers are alkyl methicone copolyols and alkyl dimethicone copolyols, in particular cetyl dimethicone copolyol, lauryl methicone copolyol, W/O emulsifiers such as sorbitan stearate, glyceryl stearate, glycerol stearate, sorbitan oleate, lecithin, glyceryl isostearate, polyglyceryl-3 oleate, polyglyceryl-3 diisostearate, PEG-7 hydrogenated castor oil, polyglyceryl-4 isostearate, acrylate/C₁₀₋₃₀ alkyl acrylate crosspolymer, sorbitan isostearate, poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoylpolyglyceryl-3 diisostearate, glycol distearate, and polyglyceryl-3 dipolyhydroxy stearate.

Component F) is preferably compounds having exactly one isocyanate-reactive group or compounds having more than one isocyanate-reactive group, where only one of the isocyanate-reactive groups reacts with the isocyanate groups present in the reaction mixture under the reaction conditions chosen.

The isocyanate-reactive groups of component F) may be any functional group that can react with an isocyanate group, for example hydroxyl groups, thiol groups or primary and secondary amino groups.

Isocyanate-reactive groups in the context of the invention are especially preferably primary or secondary amino groups that react with isocyanate groups to form urea groups. As well as the amino group, compounds of component F) may also have other groups that are in principle isocyanate-reactive, for example OH groups, where just one of the isocyanate-reactive groups reacts with the isocyanate groups present in the reaction mixture under the reaction conditions chosen. This can be accomplished, for example, by reaction of the appropriate aminoalcohols at relatively low temperatures, for example at 0 to 60° C., preferably at 20 to 40° C. Preference is given to operating in this case in the absence of catalysts which would catalyze the reaction of isocyanate groups with alcohol groups.

Examples of suitable compounds of component F) are primary/secondary amines, such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, diethanolamine, 3-amino-1-methylaminopropane, 3-amino-1-ethylaminopropane, 3-amino-1-cyclohexylaminopropane, 3-amino-1-methylaminobutane, 6-aminohexanoic acid, alanine, aspartic acid, glutamic acid, glutamine, glycine, ethanolamine, 3-aminopropanol, neopentanolamine or mixtures of at least two of these.

Suitable monofunctional compounds are also ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferably, the preferably amorphous polyurethane urea is prepared using ≥0.1% and ≤20% by weight of component F) and particularly preferably ≥0.3% and ≤10% by weight of component G), based in each case on the total mass of the preferably amorphous polyurethane urea.

Preferably, component G) is used and the molar ratio of component F) to component G) is preferably from 5:1 to 1:5, particularly preferably from 1.5:1 to 1:4 and especially preferably from 1:1 to 1:3.

Preferably, the preferably amorphous polyurethane urea is prepared using components A) to G) in the following amounts, where the individual amounts always add up to 100% by weight:
5% to 40% by weight component A),
55% to 90% by weight component B),
0.5% to 20% by weight of the sum total of components C1) and optionally E),
0.1% to 10% by weight component C2),
0% to 20% by weight component D),
0.1% to 20% by weight component F) and
0% to 10% by weight component G).

Preferably, the preferably amorphous polyurethane urea is prepared using components A) to G) in the following amounts, where the individual amounts always add up to 100% by weight:
10% to 35% by weight component A),
60% to 85% by weight component B),
1% to 15% by weight of the sum total of components C1) and optionally E),
0.5% to 4% by weight component C2),
0% to 10% by weight component D),
0.3% to 10% by weight component F) and
0.1% to 3% by weight component G).

The hairstyle-stabilizing composition preferably comprises a polyurethane urea which is obtainable by reacting at least
A) one preferably aliphatic polyisocyanate component having an average isocyanate functionality of ≥1.8 and ≤2.6, selected from HDI, IPDI and/or H12-MDI or modification products thereof,
B) one polymeric polyol component preferably comprising carboxyl or carboxylate groups,
C) one amino-functional chain extender component having at least 2 isocyanate-reactive primary and/or secondary amino groups, comprising at least one amino-functional compound C1) that does not have any ionic or ionogenic groups and/or an amino-functional compound C2) that has ionic or ionogenic groups,
D) optionally further hydrophilizing components different from C2),
E) optionally hydroxy-functional compounds having a molecular weight of 62 to 399 g/mol,
F) optionally a compound having exactly one isocyanate-reactive group or one compound having more than one isocyanate-reactive group, where only one of the isocyanate-reactive groups reacts with the isocyanate groups present in the reaction mixture under the reaction conditions chosen, and
G) optionally one aliphatic polyisocyanate component having an average isocyanate functionality of >2.6 and ≤4, where component G) consists of an aliphatic or cycloaliphatic polyisocyanate oligomer having isocyanurate, urethane, allophanate, biuret, iminooxadiazinedione or oxadiazinetrione structure.

The hairstyle-stabilizing composition preferably comprises a preferably amorphous polyurethane urea which is obtainable by reacting at least
A) one preferably aliphatic polyisocyanate component which is a mixture of IPDI and HDI,
B) one polymeric polyether polyol component which is a mixture of at least two poly(propylene glycol) polyether polyols and where the poly(propylene glycol) polyether polyols differ in their number-average molecular weights,
C) one amino-functional chain extender component having 2 isocyanate-reactive primary and/or secondary amino groups, comprising at least one amino-functional compound C1) that does not have any ionic or ionogenic groups and/or an amino-functional compound C2) that has ionic or ionogenic groups,
D) optionally further hydrophilizing components, that are different from C2), which are non-ionically hydrophilizing components D1),
E) optionally hydroxy -functional compounds having a molecular weight of 62 to 399 g/mol,
F) optionally one compound having exactly one isocyanate-reactive group or one compound having more than one isocyanate-reactive group, where only one of the isocyanate-reactive groups reacts with the isocyanate groups present in the reaction mixture under the reaction conditions chosen, where the isocyanate-reactive group is a primary and/or secondary amino and/or hydroxyl group, and

G) optionally one aliphatic polyisocyanate component having an average isocyanate functionality of >2.6 and ≤4, where component G) consists of an aliphatic or cycloaliphatic polyisocyanate oligomer having isocyanurate, urethane, allophanate, biuret, iminooxadiazinedione or oxadiazinetrione structure, based on HDI, IPDI and/or H12-MDI.

Preferably, the hairstyle-stabilizing composition is suitable for treatment of hair, especially for conserving the structure of the change in color and/or shape of the hair. In particular, the treatment of the hair with the hairstyle-stabilizing composition constitutes a further hair cosmetics application. The further hair cosmetics application may be any application to the hair that leads to a change in the color and/or shape of the hair. What is achieved by the treatment of the hair with the composition of the invention is preferably that the hair has higher structural resistance to water, for example in the form of a jet of water, for example when showering, bathing or swimming, or haircare products such as shampoo, hair fixatives, etc., low swelling and good tactile properties. Preferably, the hair cosmetics application is selected from the group consisting of
(A) dyeing at least a part of the hair,
(B) permanent wave treatment of the hair,
(C) treatment with a curling wand,
(D) straightening of the hair,
(E) permanent straightening of the hair,
(F) dressing of the hair,
(G) hair extension,
(H) hair shortening,
(I) a combination of at least two applications selected from (A) to (G).

The treatment of hair with the hairstyle-stabilizing composition according to the invention preferably takes place using elevated temperature. The employment of elevated temperature may relate either to the hair to be treated or to the hairstyle-stabilizing composition to be used, or to both. Preferably, the employment of elevated temperature selected from the group consisting of heating of the hairstyle-stabilizing composition before application to the hair, heating of the hair before treatment with the hairstyle-stabilizing composition, heating of the hair after treatment with the hairstyle-stabilizing composition, heating of the hair during treatment with the hairstyle-stabilizing composition or a combination of at least two of these, takes place. Preferably, the employment of elevated temperature takes place by heating the hairstyle-stabilizing composition or heating the hair before treatment of the hair with the hairstyle-stabilizing composition. The employment of elevated temperature takes place either before, during and/or after the treatment of hair with the hairstyle-stabilizing composition according to the invention. Preferably, the hair to be treated, during employment of elevated temperature, is contacted with a composition having a temperature in a range from 30 to 250° C., or preferably in a range from 40 to 230° C., or preferably in a range from 50 to 200° C., or preferably in a range from 60 to 180° C. The heating of the hairstyle-stabilizing composition preferably takes place before treatment of the hair therewith, preferably in a range from 30 to 100° C., or preferably in a range from 35 to 90° C., or preferably in a range from 40 to 80° C. Preferably, the elevated temperature is imparted to the hair via heated air or a heated surface. The elevation of the temperature is preferably achieved by a means selected from the group consisting of a hairdryer, a straightener, a curling iron, a hood dryer, a curling wand, or a combination of at least two of these. The specified temperature ranges for the elevated temperature is preferably the temperature of the means immediately before it is brought into contact with the hair. The heating of the hairstyle-stabilizing composition can also be effected by heating the hair prior to treatment with the hairstyle-stabilizing composition, especially if the hair is treated with a means having a temperature of more than 100° C.

The duration of the employment of elevated temperature is preferably within a range from 1 second to 3 hours, or preferably within a range from 5 seconds to 2 hours, or preferably within a range from 10 seconds to 1 hour, or preferably within a range from 20 seconds to 50 minutes. Depending on the type of hairstyle design, for example straightening of the hair or curling of the hair, the elevated temperature can be employed for different lengths of time and at different temperatures. For instance, when straightening hair, it is preferably to employ a temperature above 200° C. several times, preferably 2 to 10 times for a few seconds, preferably 2 to 30 seconds, or preferably 3 to 10 seconds. On the other hand, it is preferable to keep the temperature in a range of 50 to 100° C. when curling hair and to employ the temperature within a range from 10 to 120 minutes, or preferably within a range from 30 to 100 minutes.

The polycarbodiimide component (V2) is produced from aliphatic or cycloaliphatic polyisocyanates.

The aliphatic or cycloaliphatic polyisocyanates are preferably selected from the group consisting of methylene diisocyanate, dimethylene diisocyanate, trimethylene diisocyanate, tetramethylene diisocyanate, pentamethylene diisocyanate, dipropyl ether diisocyanate, 2,2-dimethylpentane diisocyanate, 3-methoxyhexane diisocyanate, octamethylene diisocyanate, 2,2,4-trimethylpentane diisocyanate, nonamethylene diisocyanate, decamethylene diisocyanate, 3-butoxyhexane diisocyanate, 1,4-butylene glycol dipropyl ether diisocyanate, thiodihexyl diisocyanate, metaxylylene diisocyanate, paraxylylene diisocyanate, tetramethylxylylene diisocyanate, dicyclohexylmethane 4,4′-diisocyanate (H12MDI), isophorone diisocyanate (IPDI), hexamethylene diisocyanate (HDI), hydrogenated xylylene diisocyanate (H6XDI), 1,12-diisocyanate dodecane (DDI), norbornane diisocyanate (NBDI) and 2,4-bis(8-isocyanatoctyl)-1,3-dioctylcyclobutane (OCDI) or a mixture of at least two of these. Particular preference is given to isophorone diisocyanate (IPDI), hexamethylene diisocyanate (HDI), hydrogenated xylylene diisocyanate (H₆XDI), dicyclohexylmethane 4,4′-diisocyanate (H12MDI).

Most preferably, the cycloaliphatic polyisocyanate is dicyclohexylmethane 4,4′-diisocyanate (H12MDI).

[A11] In a preferred embodiment of the composition, the polycarbodiimide component (V2) includes polycarbodiimide compounds, comprising at least three carbodiimide groups, constructed corresponding to the formula (I),

R⁴—[R¹—N═C═N]ₙ—R⁵ (I),

where
n is an integer from 3 to 100, preferably from 3 to 50, or preferably from 3 to 20, or preferably from 3 to 10,
R¹ and R⁵ are each independently linear or branched, aliphatic or cycloaliphatic, optionally substituted groups having 4 to 100, or preferably from 4 to 50, or preferably from 4 to 20, carbon atoms.
R⁴ represents linear or branched, aliphatic or cycloaliphatic, optionally substituted groups having 1 to 100, or preferably from 1 to 50, or preferably from 1 to 20, carbon atoms.

Useful substituents of the R⁴ and/or R⁵ radicals in the context of the invention include numerous organic groups, for example alkyl, cycloalkyl, aryl, alkoxy, halogen, ether, thioether, thiourethane, disulfide, sulfoxide, sulfone, sulfonate, amino, aldehyde, keto, carboxylic ester, carboxylic acid, carbonate, carboxylate, cyano, alkylsilane and alkoxysilane groups, urethane groups, allophanate groups, biuret groups, urea groups and carboxylamide groups. In particular, a portion, also called a substituent, of the R¹, R⁴ or R⁵ radicals, preferably of the R⁴ and/or R⁵ radicals, is selected from the group consisting of a polyethylene glycol group, a polypropylene group, a methoxy group, an ethoxy group, a butoxy group, a methoxy polyethylene glycol group, a urethane group, an alkyl group, a cycloalkyl group, or a combination of at least two of these.

Further preferably, at least one of the R¹, R⁴ or R⁵ radicals, preferably the R⁴ and/or R⁵ radicals, has both an alkyl group and a cycloalkyl group. In addition, it is preferable that at least one of, more preferably both of, the R⁴ and R⁵ radicals has at least one of the groups, preferably at least two of the groups, selected from the group consisting of a urethane group, a polyethylene glycol group (PEG), a polypropylene group (PPG), a methoxy group, an ethoxy group, an alkyl group and a cycloalkyl group or a combination of at least two of these. In particular, it is preferable that the R⁴ and/or R⁵ radicals have an alkyl group, a cycloalkyl group, a urethane group, a PEG group and a methoxy group, preferably in exactly that sequence. Both R⁴ and/or R⁵ radicals preferably comprise at least one urethane group and at least one group which is a radical derived from a C₁ to C₃₀ alcohol or a C₁ to C₃₀ monoalkoxy ethylene glycol.

The polycarbodiimide component, comprising at least three carbodiimide groups, also referred to hereinafter as polycarbodiimide, preferably comprises water-soluble or water-dispersible compounds.

The polycarbodiimide present in the composition according to the invention preferably has a molecular weight M_{w} (determined by GPC) in a range from 300 to 500 000 g/mol, or preferably in a range from 500 to 300 000 g/mol, or preferably in a range from 1000 to 200 000 g/mol.

A preferred process for the preparation of aqueous dispersions of polycarbodiimides comprises at least one step, wherein, in the at least one step, at least one aliphatic or cycloaliphatic polyisocyanate is converted at a temperature within the range from 160 to 230° C. in the presence of carbodiimidization catalyst to a polycarbodiimide having an average functionality greater than 3, preferably from 3 to 100, or preferably from 3 to 50, or preferably from 3 to 20, or preferably from 3 to 10, carbodiimide units. Preferably, the reaction gases are temporarily or permanently removed. The amount of carbodiimidization catalyst is preferably 50 to 3000 ppm, based on the molar amount of polyisocyanate.

For the purposes of the present invention, the average functionality of carbodiimide units means the average number of carbodiimide units. The average functionality can also be a fraction. The average functionality is preferably 3 to 50, or preferably 3 to 20, or preferably 3 to 10. The higher the functionality, the lower the dispersibility of the hydrophilized polycarbodiimide in water.

The carbodiimidization catalyst is preferably an organophosphorus compound, more preferably organophosphorus compounds selected from the group consisting of phosphine oxide, phospholane oxide and phospholene oxide, and their sulfo and imino analogs. The phosphine, phospholene and phospholane oxides, sulfides and imino derivatives can be generated in situ, inter alia, from corresponding precursors with trivalent phosphorus, such as phosphines, phospholanes and phospholenes. The phospholene oxide is preferably selected from the group consisting of 3-methyl-1-phenyl-2-phospholene 1-oxide, 3-methyl-1-ethyl-2-phospholene 1-oxide, 1,3 -dimethyl-2-phospholene 1-oxide, 1-phenyl-2-phospholene 1-oxide, 1-ethyl-2-phospholene 1-oxide, 1-methyl-2-phospholene 1-oxide. Further suitable catalysts and preferred embodiments of the process for the preparation of the polycarbodiimide are described in WO 2011/120928 A2.

A preferred process for preparing aqueous dispersions of polycarbodiimide comprises the steps of:
a) converting at least one aliphatic or cycloaliphatic polyisocyanate at a temperature in the range of 160 to 230° C. in the presence of 50 to 3000 ppm (ppm=parts per million, 1 ppm=0.0001%, molar proportions) of carbodiimidization catalyst, based on the molar amount of polyisocyanate, to a polycarbodiimide, preferably having an average functionality of 3 to 20 carbodiimide units, is reacted, wherein the reaction gases are temporarily or permanently specifically removed from the reaction medium,
b) reacting the polycarbodiimide obtained in step a) with at least one hydrophilic compound that bears at least group reactive toward isocyanate and/or carbodiimide groups, for example but not limited to those selected from the group consisting of polyethoxymonools, polyethoxydiols, polyethoxypolypropoxymonools, polyethoxypolypropoxydiols, polyethoxymonoamines, polyethoxydiamines, polyethoxypolypropoxymonoamines, polyethoxypolypropoxydiamines, hydroxyalkylsulfonates, aminoalkylsulfonates, polyethoxymono- and -dithiols, polyethoxymono- and -dicarboxylic acids, mono- and dihydroxycarboxylic acids or salts thereof to form a hydrophilized carbodiimide,
   optionally further reacting the unreacted isocyanate groups with further compounds reactive toward isocyanate groups, for example with water, alcohols, thiols, amines, mineral acids and carboxylic acids, and
c) dispersing the compound obtained in step b) in water.

Preferably, the polycarbodiimide containing isocyanate groups which is obtained in step a) is converted according to the present invention in such a way that 10 to 70 mole percent (mol %) of the isocyanate groups present in the polycarbodiimide are reacted with at least one hydrophilic compound as component step b1) of step b), where the hydrophilic compound is selected from the group consisting of polyethoxymonools, polyethoxydiols, polyethoxypolypropoxymonools, polyethoxypolypropoxydiols, polyethoxymonoamines, polyethoxydiamines, polyethoxypolypropoxymonoamines, polyethoxypolypropoxydiamines, hydroxyalkylsulfonates, aminoalkylsulfonates, polyethoxymono- and -dithiols, polyethoxymono- and -dicarboxylic acids. In component step b2) of step b), 30 to 90 mol % of the remaining isocyanate groups are then reacted with at least one compound reactive toward isocyanate groups, for example polyethoxymonools, polyethoxydiols, polyethoxypolypropoxymonools, polyethoxypolypropoxydiols, polyethoxymonoamines, polyethoxydiamines, polyethoxypolypropoxymonoamines, polyethoxypolypropoxydiamines, hydroxyalkylsulfonates, aminoalkylsulfonates, polyethoxymono-and -dithiols, polyethoxymono- and -dicarboxylic acids, water, C₁ to C₃₀ alcohols, C₁ to C₃₀ thiols, amines, mineral acids and carboxylic acids.

The polycarbodiimides are preferably reacted in component step b1) of step b) with at least one compound selected from the group of compounds conforming to the general formula (III):

R⁶—O—(CH₂—CH₂—O)ₘ—H (III)

where R⁶=C₁ to C₃₀ alkyl or acyl group and m=4 to 30,
more preferably with at least one compound selected from the group conforming to the formula (III), where R⁶ is a methyl group and m=10 to 30.

Very particular preference is given to monomethoxy polyethylene glycol with m=15-20.

Preferred C1 to C30 alcohols which can be used for further reaction of the isocyanate groups present in the polycarbodiimide that have not been fully reacted with the hydrophilic compounds in component step b2) of step b) are firstly water, low molecular weight monoalcohols, or else diols having a molecular weight of preferably from 32 to 500, more preferably from 62 to 300, g/mol. Very particular preference is given to using short-chain monoalcohols, i.e. branched or unbranched monoalcohols having 1 to 30 carbon atoms, such as methanol, ethanol, propanol, 1-butanol, 1-pentanol, 1-hexanol, cyclohexanol, cyclohexylmethanol, 2-ethylhexanol, dodecanol, stearyl alcohol or oleyl alcohol, mixtures thereof with one another and mixtures of isomers thereof and short-chain dialcohols having 2 to 60 carbon atoms, such as butane-1,4-diol, pentane-1,5-diol, hexane-1,6-diol, cyclohexanediol, cyclohexanedimethanol, octane-1,8-diol, nonane-1,9-diol, decane-1,10-diol, undecane-1,11-diol, dodecane-1,12-diol, tridecane-1,13-diol, tricyclodecanedimethanol, the mixture thereof with one another and mixtures of their isomers.

The sequence of component steps b1) and b2) of step b) may be fixed so that component step b1) may precede component step b2), they may be simultaneous or the order may be reversed.

Optionally, the aqueous dispersion of hydrophilized polycarbodiimide is adjusted in a step d) to a pH in the range of 7 to 12 (at 23° C.), more preferably in the range of 8 to 11. For this purpose, it is possible to use amine solutions, bases and conventional buffer solutions.

The composition has the compound comprising at least three carbodiimide groups, which has the general formula (II):

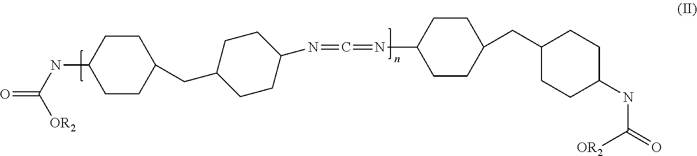

in which R₂ and R₃ are each independently a radical derived from a compound selected from the group consisting of a monoalkoxy poly(ethylene glycol) according to the general formula (III)

R⁶—O—(CH₂—CH₂—O)ₘ—H (III)
where R⁶=C₁ to C₃₀ alkyl or acyl group and m=4 to 30,
and a radical derived from C₁ to C₃₀ alcohol or a C₁ to C₃₀ monoalkoxy ethylene glycol.

The aqueous polycarbodiimide dispersions and/or solutions prepared by the process described above typically have a solids content of from 10% to 80% by weight, preferably from 20% to 60% by weight and more preferably from 30% to 50% by weight.

The hairstyle-stabilizing composition preferably comprises the compound comprising the carbodiimide, also called polycarbodiimide (V2), of the formula (I) or (III) in a range from 0.01% to 50% by weight, preferably in a range from 0.05% to 30% by weight, or preferably in a range from 0.1% to 20% by weight, or preferably in a range from 0.5% to 10% by weight, based on the total weight of the hairstyle-stabilizing composition. Depending on the use of the hairstyle-stabilizing composition, for example in hair application products, the preferred amounts of polycarbodiimides can vary widely. Hair application products are products such as hairstyling, haircare or hair coloring products. In addition, the hairstyle-stabilizing composition may also include coloring matter and a mascara, a shampoo, a setting agent, a colorant, each of which may be in the form of or be part of a spray, lotion, cream, foam, solution, emulsion or wax. The hairstyle-stabilizing composition preferably comprises the polycarbodiimide in a range from 0.1% to 10% by weight, or preferably in a range from 0.2% to 8% by weight, or preferably in a range from 0.5% to 5% by weight, based on the total weight of the hairstyle-stabilizing composition, for example in shampoos. The hairstyle-stabilizing composition preferably comprises the polycarbodiimide in a range from 0.5% to 30% by weight, or preferably in a range from 1% to 20% by weight, or preferably in a range from 2% to 15% by weight, based on the total weight of the hairstyle-stabilizing composition, in hair coloring products.

All components mentioned and described in connection with the hairstyle-stabilising composition according to the invention, their preferred amounts and their properties can be freely combined and selected.

[A12] The invention further relates to the use of at least one polyurethane urea (V1), optionally together with a polycarbodiimide (V2), for producing a hairstyle-stabilizing composition suitable for generating water-stable hairstyles, wherein the polyurethane urea (V1) is obtainable by reacting at least
A) one polyisocyanate component having an average isocyanate functionality of ≥1.8 and ≤2.6,
B) one polymeric polyol component,
C) one amino-functional chain extender component having at least 2 isocyanate-reactive amino groups, comprising at least one amino-functional compound C1) that does not have any ionic or ionogenic groups and/or an amino-functional compound C2) that has ionic or ionogenic groups,
D) optionally further hydrophilizing components different from C2),
E) optionally hydroxy-functional compounds having a molecular weight of 62 to 399 g/mol,
F) optionally a compound having exactly one isocyanate-reactive group or a compound having more than one isocyanate-reactive group, where only one of the isocyanate-reactive groups reacts with the isocyanate groups present in the reaction mixture under the reaction conditions chosen, and
G) optionally an aliphatic polyisocyanate component having an average isocyanate functionality of >2.6 and ≤4,
   wherein the polyurethane urea has at least one of the components C2) or D), and
   wherein the polyurethane urea has carboxyl groups or carboxylate groups.

What is meant by a water-stable hairstyle has already been described for the hairstyle-stabilizing composition according to the invention. All properties mentioned for the hairstyle-stabilizing composition, constituents of components, preferred proportions of components, in particular of components (V1), A) to G) and also (V2) likewise apply to the use of the polyurethane urea (V1), optionally with the polycarbodiimide (V2) as described for the hairstyle-stabilizing composition according to the invention.

[A13] The invention further relates to the use of the hairstyle-stabilizing composition according to the invention for improving the water resistance of hairstyles produced. Again, for the description and definition of water resistance, reference is made to the description of hairstyles, their production and properties in the context of the hairstyle-stabilizing composition according to the invention.

[A14] The invention further relates to a method for designing a hairstyle, in particular to water-resistant and/or wash-resistant shaping of hair into a hairstyle, comprising at least the steps of:
F1) treating the hair of a user with a hairstyle-stabilizing composition;
F2) optionally heat treating the hair of the user, wherein step F2) can take place before and/or after step F1);
F3) shaping the hair into a hairstyle;
F4) optionally drying the hair;
F5) optionally bringing the hair into contact with water before, during or after one of the steps F1) to F4), preferably before step F1);
F6) optionally repeating at least once at least part of steps F4) and F5).

Contacting the hair with water in point F5) is in the context of the invention understood as meaning that at least part of the hair shaped in step F3) is immersed in water. What is to be understood by water has already been defined above. Contacting the hair here preferably includes a washing process with surfactant-containing water, also referred to as shampooing. The treatment with the composition according to the invention in step F1) can be effected in one or in two steps. If the treatment in step F1) is in two steps, the polyurethane urea (V1) is first applied to the hair as a single component and in a subsequent step the polycarbodiimide component (V2) or vice versa. If the treatment in step F1) is carried out in one step, the two components (V1) and (V2) are already mixed. Preferably, step F5) is performed before step F1), i.e. the composition according to the invention is introduced into the wet hair.

The preferred way to design the hairstyle is to first perform step F5), then step F1), then step F3) and finally, optionally step F2) or F4). Step F3) is preferably performed together with step F3), especially if the hair is heated directly with the hairstyling agent. Advantageously, the hairstyling can now be repeated over several steps F5), especially in the form of shampooing, without having to completely redesign the hairstyle. Preferably, the hairstyle is maintained after each step F5) to an extent of at least 50%, or preferably at least 70%, or at least 90%, compared to the condition before the previous step F5).

[A15] The invention further relates to a method for producing a hairstyle-stabilizing composition comprising at least the steps of:
i. producing a polyurethane urea (V1) from an isocyanate group-containing prepolymer a., which is obtainable from the reaction of at least the components
   A) at least one polyisocyanate having a functionality of ≥1.5 to ≤6, preferably of ≥1.5 to ≤3; with
   B) at least one polyol having a functionality of ≥1.5 to ≤6, preferably of ≥1.5 to ≤3;
ii. reacting the prepolymer a. with at least
   C) a mixture C) of primary and/or secondary amino-functional compounds C1) and of primary and/or secondary diamino compounds C2) to give the polyurethane urea (V1);
iii. mixing the polyurethane urea (V1) with at least one polycarbodiimide (V2), produced from aliphatic or cycloaliphatic polyisocyanates, to obtain the hairstyle-stabilizing composition.

The polyurethane urea (V1) is preferably the polyurethane urea (V1) described in the context of the hairstyle-stabilizing composition according to the invention. The polycarbodiimide (V2) is preferably the polycarbodiimide (V2) described in the context of the hairstyle-stabilizing composition according to the invention. Preferably, all components, their properties, their preferred amounts in all mentioned combinations are also transferable to the components of the same name in the context of the method according to the invention for producing a hairstyle-stabilizing composition. For the selection and preparation of the individual components under step i. to iii., reference is also made to the description of the hairstyle-stabilising composition according to the invention. The conditions under which component A) reacts with B) in step i. are well known to the person skilled in the art. The reaction of components A) and B) is preferably carried out at a temperature in a range of 30 to 100° C., preferably in a range of 40 to 90° C. Components D) to F), if used at all, are preferably used together with component C) in step ii. Component G), if used at all, is preferably used with components A) and B) in step i.

The invention further relates to a kit, comprising at least components (V1) and (V2), in particular
(V1) a polyurethane urea which is obtainable by reacting at least
   A) one polyisocyanate component having an average isocyanate functionality of ≥1.8 and ≤2.6,
   B) one polymeric polyol component,
   C) one amino-functional chain extender component having at least 2 isocyanate-reactive amino groups, comprising at least one amino-functional compound C1) that does not have any ionic or ionogenic groups and/or an amino-functional compound C2) that has ionic or ionogenic groups,
   D) optionally further hydrophilizing components different from C2),
   E) optionally hydroxy-functional compounds having a molecular weight of 62 to 399 g/mol,
   F) optionally a compound having exactly one isocyanate-reactive group or one compound having more than one isocyanate-reactive group, where only one of the isocyanate-reactive groups reacts with the isocyanate groups present in the reaction mixture under the reaction conditions chosen, and
   G) optionally an aliphatic polyisocyanate component having an average isocyanate functionality of >2.6 and ≤4,
wherein at least one of the components C2) or D) is present in the hairstyle-stabilizing composition,
wherein the polyurethane urea dispersion comprises carboxyl groups or carboxylate groups
   and
(V2) a polycarbodiimide component produced from aliphatic or cycloaliphatic polyisocyanates.

The components, their proportions and also the method for producing components (V1) and (V2) correspond to those components A) to G) described in the context of the hairstyle-stabilising composition according to the invention. Components (V1) and (V2) are preferably processed according to the method according to the invention for producing the hairstyle-stabilizing composition according to the invention. In particular, the two components (V1) and (V2) form the composition according to the invention after their mixing. As already described above, the two components (V1) and (V2) can be mixed at least partially before, during or after styling the hairstyle. For instance, components (V1) and (V2) can be mixed and applied to the hair at a suitable time but also one of the components (V1) or (V2) can be applied before styling the hair, and the other component afterwards. It is also conceivable that both components (V1) and (V2) are applied at the same time or consecutively to the hair after styling of the hairstyle. In order to achieve a particularly long-lasting effect of hairstyle stabilization by means of the hairstyle-stabilizing composition, care should always be taken to ensure that the two components (V1) and (V2) contact or mix sufficiently. The two components (V1) and (V2) may be packaged as a kit in a common container, with the two components each provided in separate containers, or they may be packaged completely separately from each other in separate containers.

### Methods:

Unless indicated otherwise, all percentages are based on weight and the total amount or on the total weight of the compositions.

Unless stated otherwise, all analytical measurements relate to measurements at temperatures of 23° C.

Solids contents were ascertained in accordance with DIN EN ISO 3251 by heating a weighed sample at 105° C. to constant weight. At constant weight, the solids content was calculated by reweighing the sample.

Unless explicitly mentioned otherwise, NCO values were determined by volumetric means in accordance with DIN-EN ISO 11909.

The check for free NCO groups was conducted by means of IR spectroscopy (band at 2260 cm⁻¹).

The viscosities reported were determined by means of rotary viscometry to DIN 53019 at 23° C. with a rotary viscometer from Anton Paar Germany GmbH, Ostfildern, DE (1 Pa s=1 N/m²*s).

Average particle sizes (the number average is specified) of the polyurethane dispersions were determined after dilution with deionized water by means of laser correlation spectroscopy (instrument: Malvern Zetasizer 1000, Malver Inst. Limited).

The pH was measured by the method described in DIN ISO 976 on the undiluted sample.

### Experiments

For the experiments of example 1 and 2, either wavy tresses of European hair (overall length 18 cm, width 8 cm, weight 1.0 g±0.2 g) were used or straight tresses of European hair (overall length 19 cm, width 3 cm, weight 1.0 g±0.2 g). The length of the tresses corresponds to L0.

Preparation (Curling or Straightening) of Hair Tresses (step 0)):

Prior to the experiments as described in examples 1 and 2, the hair tresses were washed with 0.3 g of a commercial silicone-free shampoo (Syoss “Volume Lift Shampoo”) at 38.5° C. for 1 minute. The hair tresses were then rinsed at 38° C. for 1 minute and combed through with a conventional comb (broad side). The hair tresses were then dried with a hairdryer for 1 minute at ca. 75° C.

### Substances Used

The substances tested are abbreviated as follows:
Product A: Dispercoll U XP 2643, from Covestro Deutschland AG, aqueous polyurethane dispersion, solids content 40%, (V1)
Product B: Desmodur XP 2802, from Covestro Deutschland AG, aliphatic polycarbodiimide, solids content 40% in water, (V2)
Product C: Baycusan C1008, INCI: Polyurethane-48 from Covestro Deutschland AG, aqueous dispersion, solids content 30%
Product D: Luviflex Soft (INCI: Acrylate copolymer), from BASF AG, Germany
Product E: Amphomer (INCI: Octylacrylamide/Acrylates/Butylaminoethyl Methacrylate Copolymer), 100% with AMP (Amino Methylpropanol) adjusted to pH 9.6, 2% by weight, from Akzonobel

Product F: Syntran PC 5100 (INCI: Polyacrylate 21 (and) acrylate/Dimethylaminoethyl Methacrylate Copolymer), commercially available from the supplier Interpolymer, aqueous solution, solids content 25%

### Curl Formation:

Design of a Hairstyle: Shaping the Tresses into Curls (step 1)

A washed hair tress (European straight hair tresses, prepared as described in step 0) was placed in a bowl standing on a balance. 1g of a 2% by weight (solids content) aqueous solution was distributed over the entire length of the hair tress so that the solution was distributed as homogeneously as possible. The hair tress was then combed until it was completely wetted. The tress was then smoothed with 2 fmgers. This was then twisted on a perm rod with a gap between 2 and 3 mm. The untwisting was carried out clockwise with the help of a weight attached to the end of the tress. The hair tress was then fastened with an elastic band. All finished tresses were then hung in the 50% humidity chamber. The tresses were dried in the humidity chamber for at least 18 hours.

After drying, the tresses were unwound counterclockwise on one side. The length of the tresses was determined as LS.

### EXAMPLE 1

### High Humidity Curl Retention of Product A and Product B (Polycarbodiimide) Alone and in Mixtures

Treatment in humidity chamber (humidity test): Following the preparation of step 0), the curls were hung in a 90% humidity chamber at 30° C. The air humidity was adjusted with distilled water. The curl retention capacity was then determined using a centimeter scale, which was permanently installed in the humidity chamber and lowered down after fixed time intervals: 10 min, 20 min, 30 min, 45 min, 60 min, 2 h, 3 h, 4h, 5 h, 6 h and 24 h. By means of the 24 h value, the LT value was determined.
L0=length of the hair tress prior to any treatment
LS=length of the hair tress after step 1 but prior to the humidity test
LT=length of the curl after humidity test (here after 24 hours at 30° C. and 90% air humidity)

$$Lockenhaltenvermögen in \% = 100 \times \frac{( L 0 - LT )}{( L 0 - LS )}$$

TABLE 1
Results with hair treated with different inventive^{eg} and non-inventive^{neg}
mixtures and their curl retention capacity at high air humidity

| Example | Product A:Product B [g:g] | Curl retention capacity [%] |
|---|---|---|
| 1-a | 1:0^{neg} | 31 |
| 1-b | 0.5:0.5^{eg} | 73 |
| 1-c | 0.7:0.3^{eg} | 70 |
| 1-d | 0.9:0.1^{eg} | 47 |
| 1-e | 0:1^{neg} | <30 |

Table 1 shows examples of inventive mixtures 1-b to 1-d in comparison with non-inventive mixtures of components (V1) in the form of product A 1-a and (V2) in the form of product B 1-e. It can be clearly seen that product A alone, which is not in accordance with the invention, has a very low moisture resistance for curly hair, as shown in example 1-a. The same applies to product B alone, which is not in accordance with the invention, in example 1.e. In both examples, 1-a and 1-e, only a curl retention capacity of around or less than 30% is achieved. By using the mixture of components (V1) as product A and (V2) as product B according to the invention, the water resistance could be significantly increased, which is shown in the percentage curl retention capacity figures of 47 to 73% for the mixtures 1-b to 1-d. Furthermore, it can be seen that the curl retention capacity with a 1:1 mixture 1-b of component (V1) and (V2) has a maximum for the measured mixtures.

### EXAMPLE 2

### Curl Retention Capacity in Water

Treatment in water: After preparation as described in step 0), 1 g of solution of the mixtures 2-a to 2-c were applied to European straight hair tresses and distributed in the hair as described in step 1. Their curl retention capacity in water was then determined.
2-a^{eg}: product A: product B in the ratio 1:1.2% by weight solids content
2-b^{neg}: product D: product B in the ratio 1:1.2% by weight solids content
2-c^{neg}: product F: product B in the ratio 1:1.2% by weight solids content

To determine the curl retention capacity in water the procedure was as follows: A hair curl was dipped into a water bath at 38° C. for 30 seconds. The curl was then removed from the water bath, hung up wet and the length of the curl was immediately measured (LT wet). The curl was then fully air-dried at room temperature and the length again determined (LT dry).
L0=length of the hair tress prior to any treatment (=19 cm)
LS=length of the hair tress after treatment (curled) prior to the test
LT wet=length of the wet curl directly after 30 seconds in the water bath
LT dry=length of the curl after 30 seconds in the water bath and drying at room temperature

$$Wasserbeständigkeit ( nass ) in \% = 100 \times \frac{( L 0 - LT nass )}{( L 0 - L S )}$$

$$Wasserbeständigkeit ( trocken ) in \% = 100 \times \frac{( L 0 - LT trocken )}{( L 0 - L S )}$$

TABLE 2
Results with hair treated with different inventive^{eg} and
non-inventive^{neg} mixtures and their water treatment

| | Curl retention | Water resistance | Water resistance |
|---|---|---|---|
| Example | capacity [%] | wet [%] | dry [%] |
| 2-a^{eg} | 76 | 58 | 42 |
| 2-b^{neg} | 77 | 40 | 20 |
| 2-c^{neg} | 77 | 36 | 27 |

Although the three mixtures 2-a to 2-c result in similar curl retention capacity, the mixture 2-a (inventive) allows a significantly better water resistance, measured both with wet and dry hair.

### EXAMPLE 3

### Water Resistance and Shampoo Resistance of Straightened Hair

After the preparation as described in step 0), the wet hair tresses (European curly hair tresses (BO=4 to 5 cm width)) were treated with one of the mixtures 3-a (inventive), 3-b, or 3c (non-inventive comparative examples). The hair was then dried by hair dryer for 3 minutes at 75° C. and straightened with a commercial flat iron 5 times at 230° C. for 3 seconds and then completely dried at 56% rel. air humidity at 23° C. for 12 hours.
3-a: product A: product B in the ratio 1:1.2% by weight solids content
3-b: product C: product B in the ratio 1:1.2% by weight solids content
3-c: product F: product B in the ratio 1:1.2% by weight solids content

Water resistance: The width of the respective straightened tress was determined for the determination of BG. The tress was then dipped in a water bath at 38° C. for 30 seconds, then the tress was hung up and completely air-dried at room temperature. The width of the tress was then measured again for the determination of BW.

TABLE 3
Results with hair treated and straightened with different
inventive^{eg} and non-inventive^{neg} mixtures and their water treatment

| | Untreated width | Straightened width | Width BW after 30 sec in |
|---|---|---|---|
| Mixture | B0 [cm] | BG [cm] | water and drying [cm] |
| 3-a^{eg} | 4 | 2.5 | 2.0 |
| 3-b^{neg} | 4 | 3 | 1 |
| 3-c^{neg} | 5 | 2.5 | 5 |

Wash resistance: The width of the straightened tress was measured for the determination of BG. The tress was then washed at 38° C. for 1 minute with a commercial shampoo (Syoss Volume Lift). The hair was then rinsed with water for approximately 30 seconds. The tress was then hung up and completely dried at room temperature. Finally, the width of the tress was measured for the determination of BST.

If the tress was still straight (<4 cm), the same tress was shampooed again and the wash resistance again determined by determination of the width B2ST.

TABLE 3
Results with hair treated and straightened with different
inventive^{eg} and non-inventive^{neg} mixtures and their shampoo treatment

| | | Width after | Width after |
|---|---|---|---|
| | Straightened | 1 * shampoo and | 2 * shampoo and |
| Mixture | width BG [cm] | drying BST [cm] | drying B2ST [cm] |
| 3-a^{eg} | 3 | 1.5 | 3 |
| 3-b^{neg} | 2.5 | 0.5 | 1.5 |
| 3-c^{neg} | 3 | 5 | — |

Mixture 3-c showed neither water resistance nor shampoo resistance. Mixture 2-b showed better water and shampoo resistance but the hair tended to stick together, resulting in a not very aesthetic appearance of the hair. In contrast, on straightened hair, mixture 3-a (inventive) shows very good water and shampoo resistance (even after 2 shampoo treatments) of the straightening of the hair, without the hair sticking together. In addition, hair treated with mixture 3-a felt very soft (like healthy hair) and looked very natural.

1. A hairstyle-stabilizing composition comprising:
(V1) a polyurethane urea which is obtained by reacting components of a reaction mixture, said reaction mixture comprising at least
A) one polyisocyanate component having an average isocyanate functionality of ≥1.8 and ≤2.6,
B) one polymeric polyol component,
C) one amino-functional chain extender component having at least 2 isocyanate-reactive amino groups, comprising at least one amino-functional compound C1) that does not have any ionic or ionogenic groups and/or an amino-functional compound C2) that has ionic or ionogenic groups,
D) optionally further hydrophilizing components different from C2),
E) optionally hydroxy-functional compounds having a molecular weight of 62 to 399 g/mol,
F) optionally a compound having exactly one isocyanate-reactive group or a compound having more than one isocyanate-reactive group, where only one of the isocyanate-reactive groups reacts with the isocyanate groups present in the reaction mixture, and
G) optionally an aliphatic polyisocyanate component having an average isocyanate functionality of >2.6 and ≤4,
wherein the polyurethane urea has at least one of- the components C2) or D), and
wherein the polyurethane urea has carboxyl groups or carboxylate groups; and
(V2) a polycarbodiimide component produced from aliphatic or cycloaliphatic polyisocyanates.

2. The composition as claimed in claim 1, wherein the polyisocyanate A) comprises at least one of 1,4-butylene diisocyanate, 1,6-hexamethylene diisocyanate 2,2,4-trimethylhexamethylene diisocyanate, and/or 2,4,4-trimethylhexamethylene diisocyanate, isomeric bis(4,4′-isocyanatocyclohexyl)methanes or mixtures of any isomer content thereof, 1,4-cyclohexylene diisocyanate, 1,4-phenylene diisocyanate, 2,4-toluylene diisocyanate, 2,6-toluylene diisocyanate, 1,5-naphthylene diisocyanate, 2,2′-diphenylmethane diisocyanate, and/or 2,4′-diphenylmethane diisocyanate, 4,4′-diphenylmethane diisocyanate, 1,3-bis(2-isocyanatoprop-2-yl)benzene, 1,4-bis(2-isocyanatoprop-2-yl)benzene, 1,3-bis(isocyanatomethyl)benzene, alkyl 2,6-diisocyanatohexanoate having C1-C₈-alkyl groups, 4-isocyanatomethyl-1,8-octane diisocyanate, and triphenylmethane-4,4′,4″-triisocyanate.

3. The composition as claimed in claim 1, wherein polyisocyanate component A) comprises an aliphatic or cycloaliphatic polyisocyanate.

4. The composition as claimed in claim 1, wherein the component B) comprises poly(propylene glycol) polyether polyols.

5. The composition as claimed in claim 1, wherein the component B) has an average molecular mass in a range of 400 to 4000 g/mol.

6. The composition as claimed in claim 1, wherein the component B) comprises a mixture of poly(propylene glycol) polyether polyols having number average molecular weights differing by at least 100 g/mol.

7. The composition as claimed in claim 1, wherein the polyol B) comprises at least one of succinic acid, methylsuccinic acid, glutaric acid, adipic acid, pimelic acid, suberic acid, azelaic acid, sebacic acid, nonanedicarboxylic acid, decanedicarboxylic acid, terephthalic acid, isophthalic acid, o-phthalic acid, tetrahydrophthalic acid, hexahydrophthalic acid, cyclohexanedicarboxylic acid, maleic acid, fumaric acid, malonic acid, trimellitic acid, o-phthalic anhydride, trimellitic anhydride, succinic anhydride, and mixtures thereof with a polyhydric alcohol.

8. The composition as claimed in claim 1, wherein the component C) comprises at least one amino-functional compound C1) having no ionic or ionogenic groups and an amino-functional compound C2) which has ionic or ionogenic groups.

9. The composition as claimed in claim 1, wherein the component D) comprises exclusively non-ionically hydrophilizing components D1).

10. The composition as claimed in claim 1, wherein the polycarbodiimide component (V2) includes polycarbodiimide compounds, comprising at least three carbodiimide groups, corresponding to the formula (I),
R⁴—[R¹—N═C═N]ₙ—R⁵ (I),
wherein
n is an integer from 3 to 100,
R¹ and R⁵ are each independently linear or branched, aliphatic or cycloaliphatic, optionally substituted groups having 4 to 100 carbon atoms, and
R⁴ represents linear or branched, aliphatic or cycloaliphatic, optionally substituted groups having 1 to 100, carbon atoms.

11. (canceled)

12. (canceled)

13. A method for styling a hairstyle with at least the steps of:
F1) treating the hair of a user with a hairstyle-stabilizing composition as claimed in claim 1;
F2) optionally heat treating the hair of the user, wherein step F2) is performed before and/or after step F1);
F3) shaping the hair into a hairstyle;
F4) optionally drying the hair;
F5) optionally bringing the hair into contact with water;
F6) optionally repeating at least once at least part of steps F4) and F5.

14. A method for producing a hairstyle-stabilizing composition, comprising:
i. producing a polyurethane urea (V1) from an isocyanate group-containing prepolymer a., which is obtained from a reaction of at least the components
A) at least one polyisocyanate having a functionality of ≥1.5 to ≤6; with
B) at least one polyol having a functionality of ≥1.5 to ≤6;
ii. reacting the prepolymer a. with at least
C) a mixture C) of primary and/or secondary amino-functional compound C1) and of primary and/or secondary diamino compounds C2) to give the polyurethane urea (V1);
iii. mixing the polyurethane urea (V1) with at least one polycarbodiimide (V2) to obtain the hairstyle-stabilizing composition, wherein the polycarbodiimide (V2) is produced from aliphatic or cycloaliphatic polyisocyanates.

15. A kit, comprising at least the components:
(V3) a polyurethane urea which is obtained by reacting components of a reaction mixture, the reaction mixture comprising at least
A) one polyisocyanate component having an average isocyanate functionality of ≥1.8 and ≤2.6,
B) one polymeric polyol component,
C) one amino-functional chain extender component having at least 2 isocyanate-reactive amino groups, comprising at least one amino-functional compound C1) that does not have any ionic or ionogenic groups and/or an amino-functional compound C2) that has ionic or ionogenic groups,
D) optionally further hydrophilizing components different from C2),
E) optionally hydroxy-functional compounds having a molecular weight of 62 to 399 g/mol,
F) optionally a compound having exactly one isocyanate-reactive group or one compound having more than one isocyanate-reactive group, where only one of the isocyanate-reactive groups reacts with the isocyanate groups present in the reaction mixture, and
G) optionally an aliphatic polyisocyanate component having an average isocyanate functionality of >2.6 and ≤4,
wherein at least one of the components C2) or D) is present,
wherein the polyurethane urea dispersion comprises carboxyl groups or carboxylate groups
and
(V4) a polycarbodiimide component produced from aliphatic or cycloaliphatic polyisocyanates.
